(19) United States
(12) Patent Application Publication
(10) Pub. No.: US 2005/0154326 A1
(43) Pub. Date: Jul. 14, 2005

(54) Method and apparatus for integral evaluation and/or correction of state of organism regulation system

(75) Inventors: Alexander V. Martynenko, Kharkiv (UA); Kevin L. Ockunzzi, Brooklyn, OH (US); Mykola I. Yabluchanskiy, Kharkiv (UA); Vyacheslav I. Shulgin, Kharkiv (UA)

Correspondence Address: Christopher B. Fagan, Esq.;Fay, Sharpe, Fagan, Minnich & McKee, LLP, Seventh Floor, 1100 Superior Avenue, Cleveland, OH, 44114-5582, US

(73) Assignee: Micromedica Technologies LLC

(21) Appl. No.: 11/031,884

(22) Filed: Jan. 7, 2005

(30) Foreign Application Priority Data
Jan. 12, 2004 (RU) 2004100969
Jan. 8, 2004 (UA) 2004010059

(51) Int. Cl. A61B 5/04

(52) U.S. Cl. 600/515

(57) ABSTRACT

A method and apparatus for integral evaluation of a state of a regulation system of a subject organism is provided. In one embodiment, the method includes: a) registering a heart interval RR, b) measuring a succession of heart intervals RR to determine an integral approximation of a heart rate variability, c) performing a spectral decomposition on the succession of heart intervals RR to develop a spectrogram, d) fragmenting the spectrogram into low frequency, medium frequency, and high frequency ranges, and e) determining a first state of the regulation system by determining an index based at least in part on a capacity of the designated frequency ranges for the subject organism and corresponding predetermined average capacities of the designated frequency ranges for a like organism. In one embodiment, the apparatus includes: a plurality of sensors and a signal processing block. In another embodiment, the apparatus also includes a visualization device.

### CROSS-REFERENCE TO RELATED APPLICATIONS

This application is related to Martynenko et al. Attorney Docket No. MIMT 2 00002, entitled “Method and Apparatus for Correction of Functional State of Person,” filed Jan. 7, 2005, commonly assigned to Micromedica Technologies LLC and incorporated herein by reference.

This application claims the foreign priority benefits of Ukrainian Patent Application Ser. No. 2004010059, filed on Jan, 8, 2004, and Russian Patent Application Ser. No. 2004100969, filed on Jan. 12, 2004. The disclosure of both of these patent applications is incorporated herein by reference.

### BACKGROUND

This present exemplary embodiments relate to medicine and medical technique. They find particular application in conjunction with methods and devices for the integral evaluation and correction of regulation systems of a human organism basically and can be used for examination and prediction of evolution of a health state and selection of the optimal ways of correction, including curative and preventive actions and qualitative and quantitative estimation of efficiency, and will be described with particular reference thereto. However, it is to be appreciated that the present exemplary embodiments are also amenable to other like applications.

By way of background, the regulation of an organism is a system of multicircuit multilevel hierarchic nonlinear control. The quickest link of the control is an autonomic nervous system (ANS). It controls the functions of internal, blood and lymphatic vessels, unstriped muscles, and partially transversal striated muscles. The highest level of ANS—the highest vegetative centers—is situated in the oliencephalon on the level of the third cerebral ventricle and performs the function of ANS-somatic and ANS-motivational integration. ANS has the representations in motor, premotor and orbital zones of the cortex. The hypothalamus is situated below level and is connected with the cortex, the vegetative centers of the brain stem, and the spinal cord and controls unconditioned and conditioned reflex regulation of vitally important functions (e.g., breathing, blood circulation, metabolic tracts, etc).

The vegetative centers of the brainstem are mesoencephalitic and bulbar vegetative centers. The bulbar vegetative center among others causes vagus nerves which are the part of the parasympathetic nervous system (PSNS). The vegetative centers of the spinal cord are the thoracolumbar and the sacral vegetative centers.

Heart is directly innervated by the vagus nerve from the bulbar vegetative center and by sympathetic nerves from the thoracolumbar vegetative center. The vegetative centers of the horacolumbar and the sacral part of the spine are situated directly in its lateral horns and the thoracolumbar vegetative center forms an initial part of the sympathetic nervous system (SNS). The sacral vegetative center forms a sacral part of PSNS. They give fibers which go out of the spine among the ventral roots of the spinal nerves. The motor impulses from the stem and the spinal vegetative centers reach the organs, which they effect, along the dual neuronic tract. The first neurons are situated in the very centers and the secondary neurons are situated in the peripheral vegetative nodes. The processes of the first neurons are called preganglionic and end up on the secondary neurons. The processes of the secondary neurons go to the organs, which they effect, and are called postganglionic.

The peripheral vegetative nodes of PSNS are situated either close to the organs they effect or right on their wall. The peripheral vegetative nodes of SNS are represented in chains on both sides of the spinal cord and form the right and the left border sympathetic trunks. The sympathetic innervation of the organs it effects starts right from this level. The parasympathetic innervation is not so extensive as the sympathetic innervation. Some of the organs have double innervation, others have only the sympathetic innervation. SNS is a part of the sympathoadrenal system which additionally includes a cerebral layer of the adrenal glands and other accumulations of chromaffin cells. Many chromaffin cells are also situated in the heart.

The stimulation of SNS in its projections on the blood circulation leads to an increase of strength and frequency of heart beats, an increase of excitement conduction speed along the conductive heart system and the retractive myocardium, an increase of blood pressure, and causes vasodilation of the heart vessels and vasoconstriction of the vessels of other organs. The sympathetic influence on the heart is mediated by the release of adrenaline and noradrenaline with activation of β-adrenergic receptors. The final result is an acceleration of the slow diastolic repolarization.

The stimulation of PSNS is revealed by inverse effects. Its heart rate effects are mediated by the release of an acetylcholine. The ventricles function of a human being is mainly controlled by sympathetic nerves, the auricles function and the sinus node function are controlled by the sympathetic and the parasympathetic nerves. The vessels are liable to the sympathetic innervation. PSNS does not affect them directly but the multilevel bonds of both subsystems of ANS provide an indirect effect of PSNS on blood pressure and vascular tone. Medium- and short-term components of heart rate variability (HRV)—seconds, minutes, dozens of minutes—are connected with PSNS and SNS. PSNS and SNS innervation of the different heart parts is heterogeneous and asymmetric.

The current activity of PSNS and SNS is the result of the system mechanisms' reaction of the multicircuit and the multilevel regulation. The PSNS tone predominates in a quiescent state and the heart periods' variations considerably depend on a vagal modulation. The predominance of PSNS effects over SNS can be explained by two independent mechanisms: the cholinergically induced reduction of noradrenaline release as a response to the sympathetic stimulation and the cholinergic suppression of the response to an adrenergic stimulus.

The hormonal system, the angiotensin-renin system, the kallikrein-kinin system and some other systems are most studied among the humoral systems. Their effect on HRV in comparison with PSNS and SNS is long term and makes up minutes and hours.

The neurohumoral mechanisms of transmission—target organs—including blood circulation organs are open to not only internal but also external effects through the sense organs controlled by the central nervous system and further.

An interface of the regulatory systems with the heart is implemented on different levels but its conducting system is the basic one and is represented by two nodes and many fibers. The control of the conductive system is implemented through an interaction of synoatrial and atrioventricular nodes with the vegetative sympathetic and parasympathetic nerves, heart nerves and humoral factors as well. The basic node in physiologic conditions is a synoatrial node which is situated above the right auricle close to the place of flowing of superior vena cava into the right atrium. A wave of activation from the synoatrial node spreads along the fibers of the conductive system to the myocardium of auricles and further through the atrioventricular node to the myocardium of ventricles. High speed spreading of the activation along the fibers of the conductive system and its divaricate structure provides almost instantaneous covering of the myocardium of auricles and ventricles by the wave of activation. The information about myocardium dynamics of the auricles and the ventricles is used by the systems of control through the mechanoreceptors which are situated in the chambers' walls. Some receptors react to compression, other receptors react to the walls' distention. Due to this the selectivity of information in the corresponding phases of the heart cycle and its reliability is provided. There are more receptors in the auricles than in the ventricles.

One of the mechanisms of the quick regulation, which is realized in the process of several minutes, is fulfilled on the basis of the information which comes from the distension mechanoreceptors which are situated in the atrium walls. If the degree of the atriums walls distension decreases because of decrease of the blood flow, the system mechanisms which are aimed at its increase start to act. An increase of the sympathetic tone and an increase of vasopressin secretion by hypophysis which decreases renal diuresis and thus increases blood circulation volume (BCV) are among these mechanisms. Another effective mechanism of BCV maintenance is fulfilled on the regulation level of its volume in tissues through the capillary network and is based on the interaction of hydrostatic and oncotic blood pressure and intertissue liquid phase.

The speed of volume and pressure alterations in heart chambers as well as their values are important for arising of the reflex from the mechanoreceptors.

The information from the mechanoreceptors is processed in the centers of vegetative regulation and is used for formation of the control signals which are sent to the heart. Frequency and strength alteration of heart beats changes hemodynamical heart effects and thereby the state of the blood circulation on the whole. The regulatory effect on capacity of the heart is exceptional. For example, strong irritation of the mechanoreceptors of carotid arteries can bring on short-term arrest with loss of consciousness because of sharp decrease of the cerebral perfusion. The humoral link of heart regulation is biologically active substances synthesized by the specialized organs, tissues and cells which are delivered to the myocardium by liquid mediums including blood flow and intercellular ultracirculation. A number of active substances, in addition to those mentioned above, synthesize directly in the heart tissue (e.g., atrial natriuretic hormone, components of the renin-angiotensin-aldosterone system, cytokines, etc). They take part not only in the heart regulation but also in the whole system of blood circulation. The regulation mechanism, which is implemented through synthesis changes and secretion of atrial natriuretic peptide with which renal diuresis increases and BCV decreases, fulfills in the course of dozens of minutes, even hours.

The mechanoreceptors (i.e., baroreceptors and volumoreceptors) are situated in the blood vessels' walls and in the heart wall and perceive the changes of their geometry and transmit information about their current state to the regulatory centers. The aortic wall and the carotid wall are the places of the largest accumulation of the mechanoreceptors. There are also many mechanoreceptors in the large venous vessels. The mechanoreceptors react on values of average blood and venous pressure (VP), blood filling (i.e., blood volume), and other mechanic vessel characteristics as well as on the speed and the amplitude of their pulse changes. Differentiated information from the mechanoreceptors goes along the nerve trunks to the vasomotor center. The nervous mechanisms connect the blood vessels of different level of divarications with each other, with the heart and other systems and in this way coordinate their activity on the whole. Thus, it is considered that the tone change of peripheral arteries, which provide vascular resistance (VR) of blood, takes place due to the information from the receptors of the first parts of the aorta. The mechanoreceptors adapt to the steady level and range of vibration of VP and blood filling every time and that is why they react less to its changes in quasistationary conditions of rest and stress than in conditions of transient processes. Every transient process (i.e., any kind of dynamic stress) readapts the mechanoreceptors to new conditions. That is why, for example, in conditions of the arterial hypertension (AH) when the level of blood pressure (BP) becomes a steady high level, the mechanoreceptors rearrange themselves for it and take the pharmacologic hypertension as its decrease (transient process, dynamic hypotensive stress) which results in corresponding reactions as to its returning to a new steady higher level. A reflection symmetric situation takes place during the arterial hypotension. In conditions of the mechanoreceptors blockade, the vibration range of hemodynamic functions and their reflective indexes (i.e., for simpler observation—HR and VP) increase considerably and adaptation for stress decreases considerably. For example, in diastolic arterial hypotension of elderly people, when the mechanoreceptors get involved in the atherosclerotic process, the sensitivity of the mechanoreceptors decreases.

The mechanoreceptors relate to the fast-acting mechanisms of regulation. They are supported and, to some extent, duplicated by the chemoreceptors which particularly control oxygen tension in blood. If the reason for an oxygen tension decrease is a blood flow drop, a corresponding signal activates regulatory mechanisms which correct the situation properly. Except for the mechanoreceptors and the chemoreceptors importance has the action of hormones, of blood peptides and of nervous mediators, which are released from the endings of the sympathetic and parasympathetic nerves. Lately, endothelium is considered to play an important part in the regulation of the arterial tone. Nitric oxide (NO₂) is the main endothelium dependant factor of the arterial tone (relaxation) (e.g., recall the dramatic vasodilating action of nitroglycerine). The direct communication between the contours is fulfilled through the nervous system (mainly the SNS) and the humoral connections. The feedback communication is fulfilled in the form of an afferent impulsation from heart baroreceptors and vessels, chemoreceptors and vast receptor zones of different organs and tissues. The baroreflex phenomenon is the main reason for HRV in the frequency range of 0.04-0.40 Hertz.

The afferent vagus stimulation leads to the reflex activation of efferent vagus activity and to the inhibition of efferent sympathetic activity. The effects of the oppositely directed reflex are mediated by the stimulation of the afferent sympathetic activity. The efferent vagus activity is also under the tonic restricting effect of the afferent cardial sympathetic activity. The efferent sympathetic and vagus impulsations, which are directed to the sinus node, are characterized by the discharge (predominantly synchronous with every heart cycle) which is modulated by the central (e.g., vasomotor and respiratory centers) and peripheric (e.g., by fluctuations of blood pressure and respiratory movements) oscillators. These oscillators generate rhythmic vibrations of the neuronal discharges which are revealed in short-term and long-term vibrations of the heart cyclic process; and an analysis of them can be used for evaluation of the functional state of the: a) central oscillators, b) sympathetic and vagus efferent activity, c) humoral factors, and d) sinus node.

Each level of control defines the characteristic cyclic vibrations process of the functions it regulates. The higher it is, the longer the periods of vibration processes are, which is stipulated by a larger amount of its elements.

The regulatory systems duplicate and as if insure each other, ensuring their high steadiness and protection. The latter ones are revealed not only in conditions of physiological stress (e.g., changes of body position, rhythm and depth of breathing, daily fluctuation of blood hormones and environment temperature, exercise stress), but also in conditions of distress and other conditions, even the hardest pathological effects.

Through the external sensors (e.g., visual, auditory, temperature, etc.) an organism regulation systems interact with a medium, thus adapting the organism to the medium. It is natural to effect the regulation through these sensors to achieve necessary changes from this regulation.

An evaluation of the state of the organism regulation systems is an important and difficult task. The regulation systems have a paramount importance when a person is healthy or sick. A good regulation system is a sign of good health and good chances for recovering or successful sickness process irrespective of its nature or substance. The poor regulation system stipulates for health problems, such as high risk of getting sick and unfavorable outcome of sicknesses ranging from chronic sickness to lethal outcomes. That is why it is necessary to maintain the highest quality of regulation of healthy people and to deal not only with pathologic processes of sick people but also to effect positively the dynamics of regulatory processes. The individuality of a person and the person's regulatory systems which are realized in health individuality and individuality of a sickness process, presupposes individuality of reactions to medical interventions as well and individuality of these interventions. Life-style, physical exercises, professional activity, and medical interventions during sicknesses are to be carried out in such a way as to optimize the state of the regulatory systems. An optimization of the regulatory systems is a powerful instrument of life quality improvement and life span increase of a person in conditions of minimal losses on the resources necessary for their implementation.

The methods of evaluation of the state of separate regulation systems of a person's organism, the methods of defining of a direction and an extent of the system's deviation parameters from the optimal values and the methods of selection of regulatory effect on this system are well developed at the present time and include the registration of heart cycles length, their spectral decomposition, defining of spectral components, evaluation of their capacity and comparison of the correlation value of the received capacities with the values which are taken as optimal for a given individual.

The problem of all of these methods is the quasistationarity of the processes which causes inconveniences of evaluation according to multitude indexes.

A method of determination of health reserves evaluation of a person is also known (see Task Force of the European Society of Cardiology and the North American Society of Pacing and Electrophysiology, Heart Rate Variability—Standards of Measurement, Physiological Interpretation, and Clinical Use, 1996, Vol. 93, pages 1643-65). This method includes 3-5 minutes heart rate records in a steady state, identification and the measurement of RR-intervals, spectral decomposition of the received succession of heart RR-intervals, division of the received spectrogram into the frequency slots of low (VLF: 0.0033-0.04 Hertz)), medium (LF: 0.04-0.15 Hertz)) and high (HF: 0.15-0.4 Hertz)) frequencies, measurement of the total capacity of the spectrum P₀ and the capacities in designated frequency intervals P_{VLF}, P_{LF} and P_{HF}, and evaluation of the regulation state of an organism according to these values. This method allows one to evaluate the state of a person's regulation systems according to the combination of indexes: P₀, P_{VLF}, P_{LF}, P_{HF}.

The shortcoming of this method is that it presupposes absolute quasistationarity of a studied record of heart rate which is practically never reached. This is why accidental mistakes often take place that cannot be evaluated. This decreases the accuracy of the method and makes it relatively useless in conditions of any effects on a person's organism. Moreover, the method does not provide evaluation of an extent and a direction of any effect on a person's organism in actual time.

The present exemplary embodiments contemplate a new and improved method and apparatus that resolves the above-referenced difficulties and others.

### BRIEF DESCRIPTION

In one aspect, a method for integral evaluation of a state of a regulation system of a subject organism is provided. In one embodiment, the method includes: a) registering a heart interval RR for the subject organism, b) measuring a succession of heart intervals RR associated with the registered heart interval RR to determine an integral approximation of a heart rate variability for the subject organism, c) performing a spectral decomposition on the succession of heart intervals RR to develop a spectrogram, d) fragmenting the spectrogram into low frequency, medium frequency, and high frequency ranges, and e) determining a first state of the regulation system of the subject organism by determining an index based at least in part on a capacity of the designated frequency ranges for the subject organism and corresponding predetermined average capacities of the designated frequency ranges for a like organism.

In another aspect, an apparatus for integral evaluation of a state of a regulation system of a subject organism is provided. In one embodiment, the apparatus includes: a plurality of sensors adapted to sense heart intervals RR associated with the subject organism and a signal processing block in communication with each of the plurality of sensors and adapted to register a heart interval RR for the subject organism, measure a succession of heart intervals RR associated with the registered heart interval RR to determine an integral approximation of a heart rate variability for the subject organism, perform a spectral decomposition on the succession of heart intervals RR to develop a spectrogram, fragment the spectrogram into low frequency, medium frequency, and high frequency ranges, and determine a first state of the regulation system of the subject organism by determining an index based at least in part on a capacity of the designated frequency ranges for the subject organism and corresponding predetermined average capacities of the designated frequency ranges for a like organism.

In still another aspect, a method for integral evaluation of a state of a regulation system of a subject person is provided. In one embodiment, the method includes: a) registering a heart interval RR for the subject person, b) measuring a succession of heart intervals RR associated with the registered heart interval RR to determine an integral approximation of a heart rate variability for the subject person, c) performing a spectral decomposition on the succession of heart intervals RR to develop a spectrogram, d) fragmenting the spectrogram into low frequency, medium frequency, and high frequency ranges, and e) determining a first state of the regulation system of the subject person by determining an index based at least in part on a capacity of the designated frequency ranges for the subject person and corresponding predetermined average capacities of the designated frequency ranges for a like person. In this embodiment, the integral approximation of the heart rate variability is based at least in part on an equation:

$$\overline{R} {\overline{R}}_{j} = {M}_{RR} + A sin ( \frac{2 π}{{M}_{RR}} R {R}_{j} ) ;$$

where \overline{R}\overline{R}ⱼ is a model value of heart interval length, RRⱼ is a measured value of heart interval length, M_{RR} is a medium value of heart interval length, and A is a coefficient that minimizes the deviation of \overline{R}\overline{R}ⱼ from RRⱼ. Additionally, the spectral decomposition includes identification of a Fourier line with a coefficient for k members of the Fourier line and is based at least in part on first equation:

$${a}_{k} = \frac{A {M}_{RR}}{π} {\sum }_{N} ( \frac{cos ( \frac{2 π k}{{T}^{{τ}_{j + 1}}} - \frac{2 π R {R}_{j}}{{M}_{R R}} )}{2 ( k {M}_{R R} - T )} + \\ \frac{cos ( \frac{2 π k}{{T}^{{τ}_{j + 1}}} + \frac{2 π R {R}_{j}}{{M}_{R R}} )}{2 ( k {M}_{R R} + T )} - \frac{T cos ( \frac{2 π k}{{T}^{{τ}_{j}}} )}{( k {M}_{R R} + T ) ( k {M}_{R R} - T )} )$$

and at least in part on a second equation:

$${b}_{k} = \frac{A {M}_{RR}}{π} {\sum }_{N} ( \frac{sin ( \frac{2 π k}{{T}^{{τ}_{j + 1}}} - \frac{2 π {RR}_{j}}{{M}_{RR}} )}{2 ( k {M}_{RR} - T )} + \\ \frac{sin ( \frac{2 π k}{{T}^{{τ}_{j + 1}}} + \frac{2 π {RR}_{j}}{{M}_{RR}} )}{2 ( k {M}_{RR} + T )} - \frac{T sin ( \frac{2 π k}{{T}^{{τ}_{j}}} )}{( k {M}_{RR} + T ) ( k {M}_{RR} - T )} )$$

where RRⱼ is a measured value of heart interval length, M_{RR} is a medium value of heart interval length, T is a temporary interval for which the spectral decomposition is done; N is a quantity of heart intervals in a decomposition section (T=M_{RR} N), τⱼ is a first time and associated with a beginning of interval RRⱼ, and τⱼ₊₁ is a second time and associated an ending of interval RRⱼ. Moreover, the capacity of the designated frequency ranges for the subject person is determined at least in part using a first equation:
W_{O}=W_{VLF}+W_{LF}+W_{HF};
where W_{O} is the total capacity for the subject person over a frequency spectrum defined by the low, medium, and high frequency ranges, W_{VLF} is the capacity for the subject person with respect to the low frequency range, W_{LF} is the capacity for the subject person with respect to the medium frequency range, and W_{HF} is the capacity for the subject person with respect to the high frequency range. Additionally, the predetermined average capacity of the designated frequency ranges for the like person is represented at least in part using a second equation:
W_{N}=W_{VLF}+W_{LF}+W_{HF};
where W_{N} is the predetermined average capacity for the like person over a frequency spectrum defined by the low, medium, and high frequency ranges, W_{VLF} is the predetermined average capacity for the like person with respect to the low frequency range, W_{LF} is the predetermined average capacity for the like person with respect to the medium frequency range, and W_{HF} is the predetermined average capacity for the like person with respect to the high frequency range. Moreover, the index for determining the first state of the regulation system for the subject person is determined at least in part using an equation:
N=[(i_{X}−i_{N})²+(ii_{X}−ii_{N})²]^{1/2}
where i_{X}=W_{VLF}/(W_{HF}+W_{LF}) for the subject person, ii_{X}=W_{LF}/W_{HF} for the subject person, i_{N}=W_{VLF}/(W_{HF}+W_{LF}) for the like person, and ii_{N}=W_{LF}/W_{HF}—for the like person.

Further scope of the applicability of the present exemplary embodiments will become apparent from the detailed description provided below. It should be understood, however, that the detailed description and specific examples, while indicating preferred embodiments of the invention, are given by way of illustration only, since various changes and modifications within the spirit and scope of the invention will become apparent to those skilled in the art.

### BRIEF DESCRIPTION OF THE DRAWINGS

The present exemplary embodiments exist in the construction, arrangement, and combination of the various parts of the apparatus, and steps of the method, whereby the objects contemplated are attained as hereinafter more fully set forth, specifically pointed out in the claims, and illustrated in the accompanying drawings in which:

FIG. 1 is a block diagram of an exemplary embodiment of an apparatus for heart interval RR registration and processing;

FIGS. 2A and 2B are physical views of an exemplary embodiment of an apparatus for heart interval RR registration and processing;

FIGS. 3A and 3B show exemplary results from measurements of volunteer A provided by an exemplary embodiment of an apparatus for heart interval RR registration and processing;

FIG. 4 shows an exemplary evaluation result of the regulation state of volunteer A on the phase plane W_{VLF}/W_{LF}−W_{LF}/W_{HF} provided by an exemplary embodiment of an apparatus for heart interval RR registration and processing;

FIGS. 5A and 5B show an exemplary RR-intervalogram of volunteer G and an exemplary spectral decomposition for the first 128 RR-intervals provided by an exemplary embodiment of an apparatus for heart interval RR registration and processing;

FIG. 6 shows an exemplary evaluation result of the regulation state of volunteer G on the phase plane W_{VLF}/W_{LF}−W_{LF}/W_{HF} provided by an exemplary embodiment of an apparatus for heart interval RR registration and processing;

FIGS. 7A and 7B show an exemplary RR-intervalogram of volunteer A before the test with physical exercises and an exemplary spectral decomposition for the first 128 RR-intervals provided by an exemplary embodiment of an apparatus for heart interval RR registration and processing;

FIGS. 8A and 8B show an exemplary RR-intervalogram of volunteer A after the test with physical exercises and an exemplary spectral decomposition for the first 128 RR-intervals provided by an exemplary embodiment of an apparatus for heart interval RR registration and processing;

FIG. 9 shows an exemplary evaluation result of the regulation state of volunteer A on the phase plane W_{VLF}/W_{LF}−W_{LF}/W_{HF} provided by an exemplary embodiment of an apparatus for heart interval RR registration and processing before the test with physical exercises;

FIG. 10 shows an exemplary evaluation result of the regulation state of volunteer A on the phase plane W_{VLF}/W_{LF}−W_{LF}/W_{HF} provided by an exemplary embodiment of an apparatus for heart interval RR registration and processing after the test with physical exercises;

FIGS. 11A and 11B show an exemplary RR-intervalogram of volunteer M before the test with listening to a musical composition and an exemplary spectral decomposition for the first 128 RR-intervals provided by an exemplary embodiment of an apparatus for heart interval RR registration and processing;

FIGS. 12A and 12B show an exemplary RR-intervalogram of volunteer M after the test with listening to a musical composition and an exemplary spectral decomposition for the first 128 RR-intervals provided by an exemplary embodiment of an apparatus for heart interval RR registration and processing;

FIG. 13 shows an exemplary evaluation result of the regulation state of volunteer M on the phase plane W_{VLF}/W_{LF}−W_{LF}/W_{HF} provided by an exemplary embodiment of an apparatus for heart interval RR registration and processing before the test with listening to a musical composition;

FIG. 14 shows an exemplary evaluation result of the regulation state of volunteer M on the phase plane W_{VLF}/W_{LF}−W_{LF}/W_{HF} provided by an exemplary embodiment of an apparatus for heart interval RR registration and processing after the test with listening to a musical composition;

FIGS. 15A and 15B show an exemplary RR-intervalogram of volunteer D before the test with controlled breathing frequency and an exemplary spectral decomposition for the first 128 RR-intervals provided by an exemplary embodiment of an apparatus for heart interval RR registration and processing;

FIGS. 16A and 16B show an exemplary RR-intervalogram of volunteer D after the test with controlled breathing frequency without modulation and an exemplary spectral decomposition for the first 128 RR-intervals provided by an exemplary embodiment of an apparatus for heart interval RR registration and processing;

FIGS. 17A and 17B show an exemplary RR-intervalogram of volunteer D after the test with controlled breathing frequency with modulation and an exemplary spectral decomposition for the first 128 RR-intervals provided by an exemplary embodiment of an apparatus for heart interval RR registration and processing;

FIG. 18 shows an exemplary evaluation result of the regulation state of volunteer D on the phase plane W_{VLF}/W_{LF}−W_{LF}/W_{HF} provided by an exemplary embodiment of an apparatus for heart interval RR registration and processing before the test with controlled breathing frequency;

FIG. 19 shows an exemplary evaluation result of the regulation state of volunteer D on the phase plane W_{VLF}/W_{LF}−W_{LF}/W_{HF} provided by an exemplary embodiment of an apparatus for heart interval RR registration and processing after the test with controlled breathing frequency without modulation;

FIG. 20 shows an exemplary evaluation result of the regulation state of volunteer D on the phase plane W_{VLF}/W_{LF}−W_{LF}/W_{HF} provided by an exemplary embodiment of an apparatus for heart interval RR registration and processing after the test with controlled breathing frequency with modulation;

FIG. 21 shows an exemplary evaluation result of the regulation state of volunteer S with the starting position of a circumference with radius W₀ according to the position of a circumference with radius W_{N} that is situated at the center of the phase plane and corresponds to physiological norms;

FIG. 22 shows an exemplary evaluation result of the regulation state of volunteer S with the position of a circumference with radius W₀ after a first test with musical effect;

FIG. 23 shows an exemplary evaluation result of the regulation state of volunteer S with the position of a circumference with radius W₀ after a second test with musical effect;

FIG. 24 shows an exemplary evaluation result of the regulation state of volunteer Z with the starting position of a circumference with radius W₀ according to the position of a circumference with the radius W_{N} that is situated at the center of the phase plane and corresponds to physiological norms;

FIG. 25 shows an exemplary evaluation result of the regulation state of volunteer Z with the position of a circumference with radius W₀ after a test with temperature effect;

FIG. 26 shows an exemplary evaluation result of the regulation state of volunteer Zₕ with the starting position of a circumference with radius W₀ according to the position of a circumference with the radius W_{N} that is situated at the center of the phase plane and corresponds to physiological norms;

FIG. 27 shows an exemplary evaluation result of the regulation state of volunteer Zₕ with the starting position of a circumference with radius W₀ after a test with walking in place.

### DETAILED DESCRIPTION

One aspect of an exemplary embodiment provides a method for integral evaluation of the state of organism regulation systems. The method may be applied to non-steady processes, has a higher accuracy than previous methods, and provides a possibility of visual display of the results of the regulation system evaluation in actual time. This is the basis for creation of the system with biological feedback.

Another aspect of an exemplary embodiment provides a method that provides a possibility of an efficiency definition of an effect that corrects the regulation systems of an organism in conditions of non-steady processes in the organism.

Still another aspect of an exemplary embodiment provides a method for correction of the regulation system of a person's organism that is applied to non-steady processes. This method has a higher accuracy than previous methods and provides a possibility of a visual display of the correction results using biological feedback.

Yet another aspect of an exemplary embodiment provides an apparatus for integral evaluation of the state of organism regulation systems. Use of the apparatus makes it possible to define an efficiency of an effect that corrects the organism regulation systems and to correct the regulation systems of the organism using biological feedback.

In one embodiment, the method for integral evaluation of the state of organism regulation systems includes: registration of heart intervals RR, measurement of a succession of the heart intervals RR, spectral decomposition of the measured succession of heart intervals RR, division of the spectrogram into frequency slots of low frequency (VLF), medium frequency (LF) and high frequency (HF), and evaluation of the organism's regulation state according to spectrum capacities in the designated frequency intervals VLF, LF and HF.

The following integral approximation of variability of heart rate (VCR) on RR-intervals may be used in the embodiment of the method being described:

$$\overline{R} {\overline{R}}_{j} = {M}_{RR} + A sin ( \frac{2 π}{{M}_{RR}} R {R}_{j} ) ;$$

Where \overline{R}\overline{R}ⱼ is a model value of the heart cycle length; RRⱼ is a measured value of the heart cycle length; M_{RR} is a medium value of the heart cycle length; A is a coefficient that minimizes the deviation of \overline{R}\overline{R}ⱼ from RRⱼ; T is a temporary interval for which the spectral decomposition is done; N is a quantity of RR-intervals in decomposition section (T=M_{RR} N); τⱼ is the time of the beginning of the interval RRⱼ; τⱼ₊₁ is the time of the ending of the interval RRⱼ.

The spectrograms are divided into the frequency slots of VLF, LF, and HF. The total capacity of the spectrum W₀ and the capacities in the designated frequency intervals W_{VLF}, W_{LF} and W_{HF} are measured and the conditions of organism's regulation according to the value of the index are evaluated using the following equation:
N=[(i_{X}−i_{N})²+(ii_{X}−ii_{N})²]^{1/2};
Where i_{X}=W_{VLF}/(W_{HF}+W_{LF}); ii_{X}=W_{LF}/W_{HF}; i_{N}=W_{VLF}/(W_{HF}+W_{LF}) and is a medium value for a person of the given age and sex; ii_{N}=W_{LF}/W_{HF} and is a medium value for a person of the given age and sex. The low frequencies are the frequencies of 0.0033-0.04 Hertz, the medium frequencies are the frequencies of 0.04-0.15 Hertz and the high frequencies are the frequencies of 0.15-0.4 Hertz.

The current state of the regulation systems N may be represented on a phase plane W_{VLF}/(W_{HF}+W_{LF})−W_{LF}/W_{HF} in the form of a circumference with radius W_{O} with moving coordinates of the center i_{X}−ii_{X} and a circumference with radius W_{N} with coordinates of the center i_{N}−ii_{N} placed at the computing origin. Presentation of the information in this manner facilitates visual evaluation of the regulation state in actual time and allows the method to be used as a correction system of the regulation systems with biological feedback.

In one embodiment, the method that can determine an efficiency for an effect that corrects organism regulation systems includes: correctional effect on an organism and evaluation of state changes of the organism regulation systems and change of correctional effect on an organism depending on the results of the evaluation. In this embodiment, the organism is influenced by a presumably effective correctional effect during an interval which is enough for display of this effect on the organism regulation systems. The change of a current state of the organism regulation systems is evaluated under such effect according to the method for integral evaluation of the state of organism regulation systems described above. An efficiency of the correctional effect is determined according to the direction and/or value of change of the state of the organism regulation systems using the embodiment of the method being described.

An effect that presumably produces a correctional effect on an organism or its part may be chosen from a variety of possible effects, including a group of effects comprising: a pharmaceutical remedy, a thermal effect, an effect by light, an acoustic effect, an effect by food, an effect by drinks, an effect by hunger, an effect by thirst, an effect by a gas medium, an effect by a liquid medium, physical exercises of an organism, emotional stress of an organism, intellectual exercise of an organism, or any combination thereof.

In one embodiment, the method for correction of the regulation systems of a person's organism includes: integral estimation of a state of the organism regulation systems, correctional effect on an organism, control of change of state of the organism regulation systems, and change of correctional regulative influence on an organism depending on the results of evaluation. In the process of correctional effect on an organism, a current state of the organism regulation systems is evaluated under such effect according to the method for integral evaluation of the state of regulation system of the organism described above. The results of the evaluation are typically provided to the person. The person may change a correctional effect using biofeedback depending on the direction and/or value of the change of the current state of the organism regulation systems.

An effect that presumably produces a correctional effect on an organism or its part may be chosen from a variety of possible effects, including a group of effects comprising: a pharmaceutical remedy, a thermal effect, an effect by light, an acoustic effect, an effect by food, an effect by drinks, an effect by hunger, an effect by thirst, an effect by a gas medium, an effect by a liquid medium, physical exercises of an organism, emotional stress of an organism, intellectual exercise of an organism, or any combination thereof.

It is reasonable to inform a person of the results of the evaluation of a current state of the person's organism regulation systems by means of visualization of the current state of the organism regulation systems. The current state of the organism regulation systems may be represented in the form of an image on the phase plane W_{VLF}/(W_{HF}+W_{LF})−W_{LF}/W_{HF} in the form of a circumference with radius W_{O} with moving coordinates of the center i_{X}−ii_{X} and a circumference with radius W_{N} with coordinates of the center i_{N}−ii_{N} placed at the computing origin.

In one embodiment, the apparatus for integral evaluation of the state of organism regulation systems includes: sensors of heart RR-intervals and a signal processing block to process the signals from the sensors of heart RR-intervals. More particularly, processing by the signal processing block includes: registration of heart RR-intervals, measurement of succession of the heart RR-intervals, and formation of a continued model of VCR. The following integral approximation of VCR on RR-intervals may be used in the embodiment of the apparatus being described:

$$\overline{R} {\overline{R}}_{j} = {M}_{RR} + A sin ( \frac{2 π}{{M}_{RR}} R {R}_{j} ) ;$$

with spectral decomposition of the received succession of RR-intervals in Fourier line with coefficients for k member of the line determined using the following equations:

$$\begin{matrix}{a}_{k} = \frac{A {M}_{RR}}{π} {\sum }_{N} ( \frac{cos ( \frac{2 π k}{{T}^{{τ}_{j + 1}}} - \frac{2 π R {R}_{j}}{{M}_{R R}} )}{2 ( k {M}_{R R} - T )} + & \\ \frac{cos ( \frac{2 π k}{{T}^{{τ}_{j + 1}}} + \frac{2 π R {R}_{j}}{{M}_{R R}} )}{2 ( k {M}_{R R} + T )} - \frac{T cos ( \frac{2 π k}{{T}^{{τ}_{j}}} )}{( k {M}_{R R} + T ) ( k {M}_{R R} - T )} ) & \\ {b}_{k} = \frac{A {M}_{RR}}{π} {\sum }_{N} ( \frac{sin ( \frac{2 π k}{{T}^{{τ}_{j + 1}}} - \frac{2 π {RR}_{j}}{{M}_{RR}} )}{2 ( k {M}_{RR} - T )} + & \\ \frac{sin ( \frac{2 π k}{{T}^{{τ}_{j + 1}}} + \frac{2 π {RR}_{j}}{{M}_{RR}} )}{2 ( k {M}_{RR} + T )} - \frac{T sin ( \frac{2 π k}{{T}^{{τ}_{j}}} )}{( k {M}_{RR} + T ) ( k {M}_{RR} - T )} ) & \end{matrix}$$

Where \overline{R}\overline{R}ⱼ is a model value of the heart cycle length; RRⱼ is a measured value of the heart cycle length; M_{RR} is a medium value of the heart cycle length; A is a coefficient that minimizes the deviation of \overline{R}\overline{R}ⱼ from RRⱼ; T is a temporary interval for which the spectral decomposition is done; N is a quantity of RR-intervals in decomposition section (T=M_{RR} N); τⱼ is the time of the beginning of the interval RRⱼ; τⱼ₊₁ is the time of the ending of the interval RRⱼ.

The spectrograms are divided into the frequency slots of VLF (0.0033-0.04 Hertz), LF (0.04-0.15 Hertz), and HF (0.15-0.4 Hertz). Measurement of total capacity of the spectrum W_{O} and capacities in the designated frequency intervals W_{VLF}, W_{LF} and W_{HF}; and calculation of the index value are evaluated using the following equation:
N=[(iₓ−i_{N})²+(ii_{X}−ii_{N})²]^{1/2};
Where: i_{X}=W_{VLF}/(W_{HF}+W_{LF}); ii_{X}=W_{LF}/W_{HF}; i_{N}=W_{VLF}/(W_{HF}+W_{LF}) and is a medium value for a person of the given age and sex; ii_{X}=W_{LF}/W_{HF} and is a medium value for a person of the given age and sex.

It is reasonable for the apparatus to provide a visualization of the index value N on the phase plane W_{VLF}/(W_{HF}+W_{LF})−W_{LF}/W_{HF} to inform a person of the results of the evaluation. The visualization may take the form of a circumference with radius W_{O} with moving coordinates of the center i_{X}−ii_{X} and a circumference with radius W_{N} with coordinates of the center i_{N}−ii_{N} ,which are placed at the computing origin. The visualization may be provided on a display driven by a signal and/or data generated by the signal processing block based on the heart RR-intervals detected by the sensors.

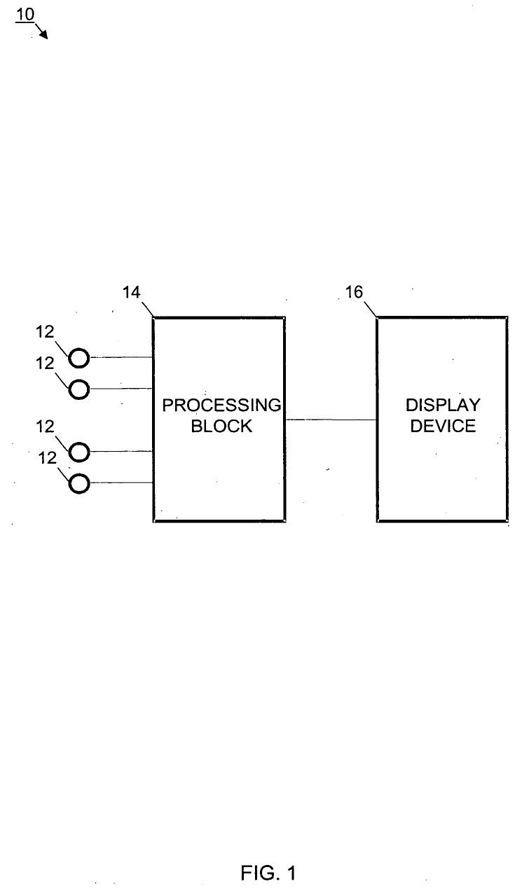

Referring now to the drawings, wherein the showings are for purposes of illustrating the preferred embodiments of the invention only and not for purposes of limiting same. FIG. 1 is a block diagram of an exemplary embodiment of an apparatus 10 for heart interval RR registration and processing. The apparatus 10 includes four sensors 12, a processing block 14, and a visualization or display device 16. The apparatus 10 is used in the examples for registration of heart intervals RR and their processing that are provided below.

The sensors 12 are adapted to sense heart intervals RR and are in communication with the processing block 14. Signals representing heart intervals RR from the sensors 12 are processed by the processing block 14 and provided to the visualization device 16. The sensors 12 of heart intervals RR, for example, are in the form of the standard electrodes for EKG recording. The processing block 14 of the signals from the sensors of the heart intervals RR includes a microprocessor that provides a possibility of the processing of the signals from the sensors of heart intervals RR according to the given algorithm. The visualization device 16 provides the results of signal processing from the sensors of heart intervals RR and may be in the form of a color monitor. One can observe a display on the visualization device 16 that characterizes a current state of the organism regulation systems.

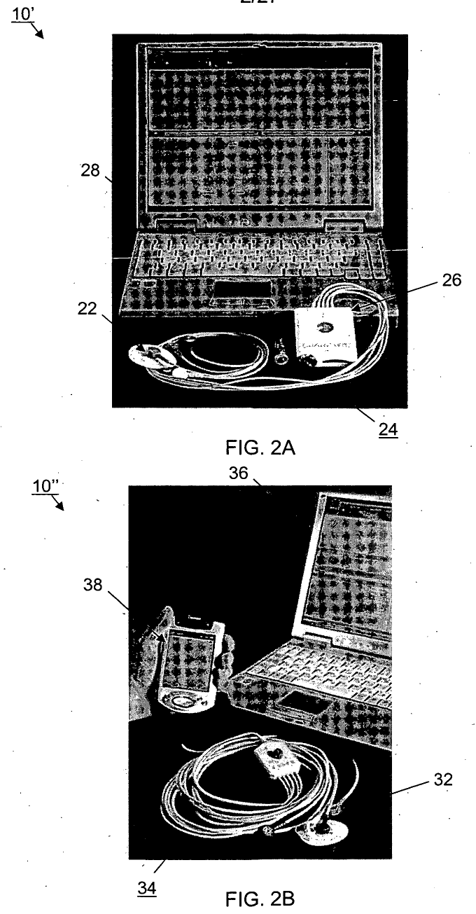

With reference to FIGS. 2A and 2B, several exemplary models of the apparatus 10′, 10″ are shown in physical views. In FIG. 2A, the sensors 12 are electrodes 22 at one end of a cable assembly 24. The other end of the cable assembly 24 provides a communication interface 26 to a notebook computer 28. The processing block 14 and visualization device 16 are provided by the notebook computer 28. In FIG. 2B, the sensors 12 are electrodes 32 at one end of a cable assembly 34. The other end of the cable assembly 34 is connected to a notebook computer 36. The notebook computer 36 is in wired or wireless communication with a personal digital assistant (PDA) 38. The processing block 14 is at least partially provided by the notebook computer 36. The PDA 38 may include a portion of the processing block 14. The visualization device 16 is provided by the PDA 38. The notebook-computer 22 may serve as a backup or redundant visualization device 16.

With reference again to FIG. 1, the sensors 12 of heart intervals are placed on a patient's body during normal operation. The sensors 12 provide signals indicative of the heart intervals to the processing block 14. The processing block performs a measurement of succession length of heart intervals, forms a continued model of VCR. The model includes an integral approximation of VCR on RR-intervals possible using the following equation:

$$\overline{R} {\overline{R}}_{j} = {M}_{RR} + A sin ( \frac{2 π}{{M}_{RR}} R {R}_{j} ) ;$$

with spectral decomposition of the received succession of RR-intervals in Fourier line with coefficients for k member of the line determined using the following equations:

$$\begin{matrix}{a}_{k} = \frac{A {M}_{RR}}{π} {\sum }_{N} ( \frac{cos ( \frac{2 π k}{{T}^{{τ}_{j + 1}}} - \frac{2 π R {R}_{j}}{{M}_{R R}} )}{2 ( k {M}_{R R} - T )} + & \\ \frac{cos ( \frac{2 π k}{{T}^{{τ}_{j + 1}}} + \frac{2 π R {R}_{j}}{{M}_{R R}} )}{2 ( k {M}_{R R} + T )} - \frac{T cos ( \frac{2 π k}{{T}^{{τ}_{j}}} )}{( k {M}_{R R} + T ) ( k {M}_{R R} - T )} ) & \\ {b}_{k} = \frac{A {M}_{RR}}{π} {\sum }_{N} ( \frac{sin ( \frac{2 π k}{{T}^{{τ}_{j + 1}}} - \frac{2 π {RR}_{j}}{{M}_{RR}} )}{2 ( k {M}_{RR} - T )} + & \\ \frac{sin ( \frac{2 π k}{{T}^{{τ}_{j + 1}}} + \frac{2 π {RR}_{j}}{{M}_{RR}} )}{2 ( k {M}_{RR} + T )} - \frac{T sin ( \frac{2 π k}{{T}^{{τ}_{j}}} )}{( k {M}_{RR} + T ) ( k {M}_{RR} - T )} ) & \end{matrix}$$

Where \overline{R}\overline{R}ⱼ is a model value of the heart cycle length; RRⱼ is a measured value of the heart cycle length; M_{RR} is a medium value of the heart cycle length; A is a coefficient that minimizes the deviation of \overline{R}\overline{R}ⱼ from RRⱼ; T is a temporary interval for which the spectral decomposition is done; N is a quantity of RR-intervals in decomposition section (T=M_{RR} N); τⱼ is the time of the beginning of the interval RRⱼ; and τⱼ₊₁ is the time of the ending of the interval RRⱼ.

The spectrograms are divided into frequency slots of VLF (0.0033-0.04 Hertz), LF (0.04-0.15 Hertz), and HF (0.15-0.4 Hertz). Measurement of total capacity of the spectrum W_{O} and capacities in the designated frequency intervals W_{VLF}, W_{LF} and W_{HF} and calculation of the index of integral evaluation of the state of the organism regulation systems are evaluated using the following equation:
N=[(i_{X}−i_{N})²+(ii_{X}−ii_{N})²]^{1/2}
Where: i_{X}=W_{VLF}/W_{LF}; ii_{X}=W_{LF}/W_{HF}; i_{N}=W_{VLF}/W_{LF} and is a medium value for a person of the given age and sex; ii_{X}=W_{LF}/W_{HF} and is a medium value for a person of the given age and sex.

An index value N on the phase plane W_{VLF}/(W_{HF}+W_{LF})−W_{LF}/W_{HF} of the monitor screen of the visualization device 16 is represented in the form of a circumference with the radius W_{O} with coordinates of the center i_{N}−ii_{N} and a circumference with the radius W_{N} with the center at the coordinate origin.

### EXAMPLE 1

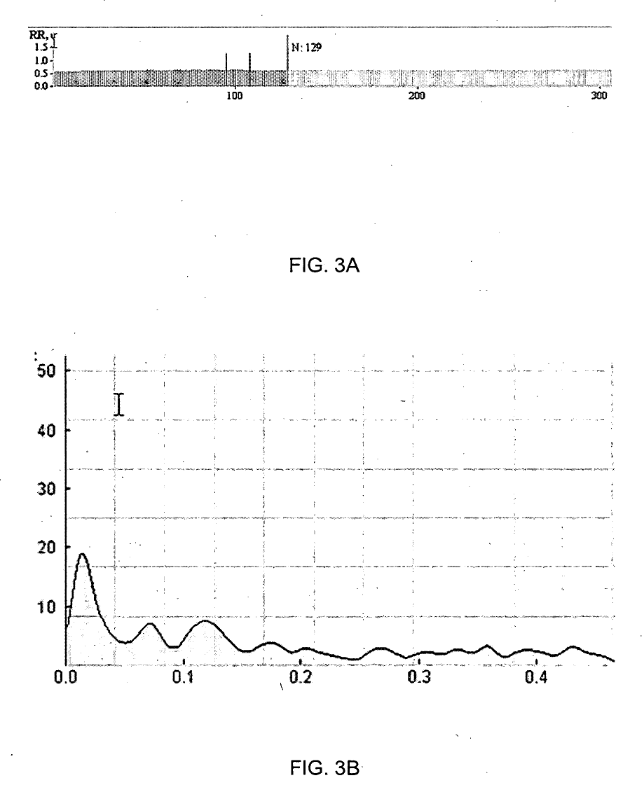

A female that was 61 years old and from Base (1) Kharkov Oblast Hospital—Agalarova, registration 16-18, was identified as patient or volunteer A. She was instructed to lay on her back on a bed in a relaxed position with her chest bare. The heart intervals RR registration apparatus 10 (FIG. 1) was used to register a succession of heart intervals RR, to measure their length, and to make an RR-intervalogram. Two electrodes of the heart intervals RR registration device were placed on her chest. The “negative” electrode was placed on the left edge of presternum and the “positive” electrode along the middle clavicular line at the level of the fifth intercostal on the left side. Next, volunteer A was asked to remain still in the above position without concentrating her attention on the process that would be performed. The registration of the succession of heart intervals RR, measurement of the length, and development of the RR-intervalogram (FIG. 3A) were accomplished using the apparatus.

The RR-intervalogram for volunteer A and the corresponding spectrum are represented in FIGS. 3A and 3B, respectively. The first 128 RR-intervals according to which the evaluation of the state of regulation systems was done are represented in FIG. 3A. The continual model of the VCR according to the first 128 RR-intervals was built. The integral approximation of VCR with a spectral decomposition of the received succession of RR-intervals, division of the spectrogram into frequency slots of VLF (0.0033-0.04 Hertz), LF (0.04-0.15 Hertz), and HF (0.15-0.4 Hertz) were fulfilled, including measurement of total capacity of the spectrum W_{O} and capacities in the designated frequency intervals W_{VLF}, W_{LF} and W_{HF}.

The results of the calculation of the indexes of the regulation systems state of the volunteer A's organism according to the declared method and prototype, as well as the average normative values for women of the corresponding age are given below for comparison:

| | | Average for women |
|---|---|---|
| Declared method | Prototype | of the given age |
| TP = 56 | 350 | 1250-2750 |
| LF/HF = 1.7 | 2.2 | 1.5-1.9 |
| VLF/LF = 2.4 | 2.1 | 1.2-1.5 |

The prototype raised was almost seven times the TP grade (total power—total power of spectrum) and its components. It is stipulated by the fact that even though volunteer A followed the instructions strictly (i.e., remained still in the desired position without concentrating her attention on the process), the RR-intervalogram showed considerable non-linearity (index of non-linearity M=1.3 with the acceptable level for standard analysis M<0.03). In other words, due to the absence of quasistationarity, the results for the prototype in this case could not be applied by any means. It should be noted that the result according to the prototype is substantially raised as a result of the non-linearity, exclusively because of the absence of artifacts of registration or any ectopic facts (extrasystoles). This example shows an existence of restrictions on the application of the prototype in the conditions of a violation of quasistationarity conditions and a possibility of the declared method application. The received data allow one to conclude that the patient has a substantial decrease of capacity and disturbance of the structure of the general regulation.

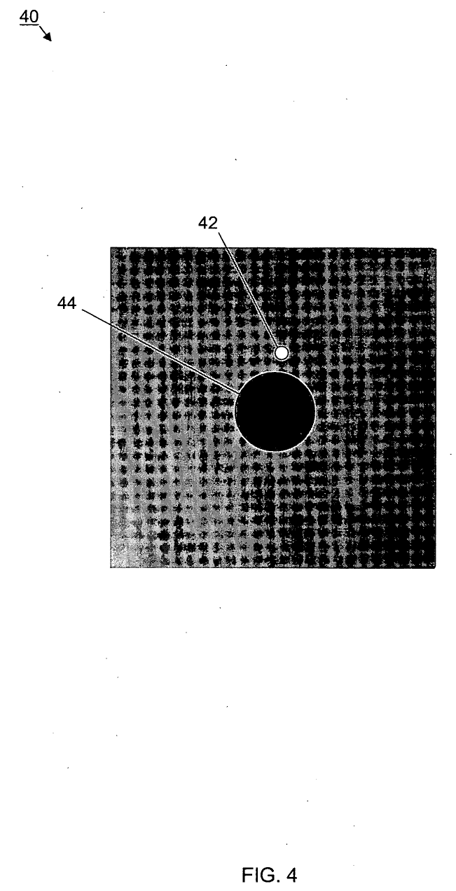

With reference to FIG. 4, the results of the evaluation of volunteer A's regulation state are represented on the phase plane W_{VLF}/W_{LF}−W_{LF}/W_{HF} 40 in the form of a circumference with the radius W₀ 42 with moving coordinates of the center i_{X}−ii_{X} and a circumference with the radius W_{N} 44 with coordinates of the center i_{N}−ii_{N} placed at the computing origin. The circumference of radius W_{N} 44 with coordinates of the center i_{N}−ii_{N} determines the normative position for women of this age and the level of regulation. The circumference of radius W₀ 42 with moving coordinates of the center i_{X}−iiX determines the regulation state of volunteer A. The distance between the centers of the circumferences and correlation of the radiuses of the circumferences show the degree of the deviation of the volunteer A's regulation state from the age norms.

As one can see from FIG. 4, the circumference of radius W₀ 42 with coordinates of the center i_{X}−ii_{X} is deviated from the circumference of radius W_{N} 44 with moving center coordinates i_{N}−ii_{N} on the axis of ordinates and has a significantly smaller normative radius. The determined condition of the patient is characterized by a substantial decrease of the regulation capacity with a disbalance in the domains capacities W_{VLF} and W_{LF}.

### EXAMPLE 2

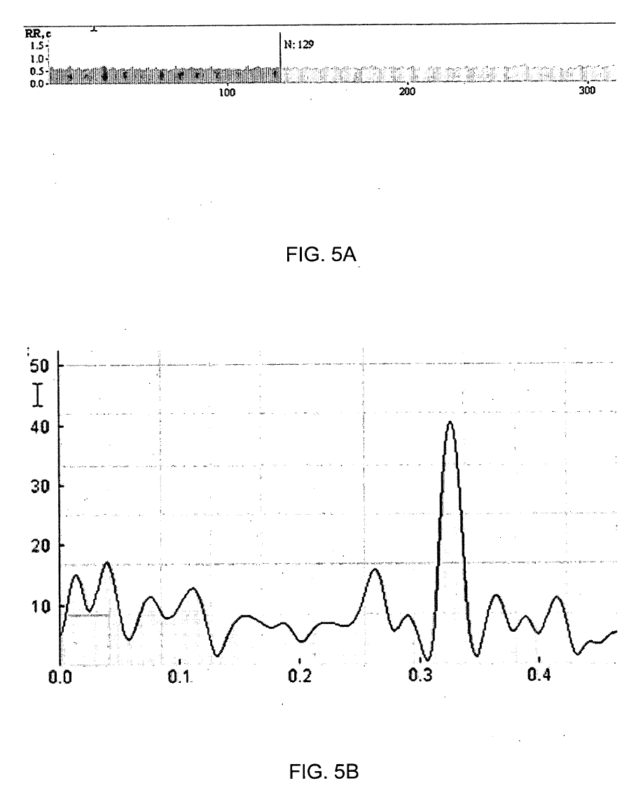

A female that was 15 years old and from Base (folder) Gorodeckaya, registration 12-26, was identified as patient or volunteer G. She was instructed to sit in an armchair in a relaxed position, leaning against the back of the armchair. Two electrodes of the heart intervals RR registration device were placed on her right and left forearms. The “negative” electrode was placed on the left forearm and the “positive” electrode was placed on the right forearm. Next, volunteer G was asked to remain still in this position for the whole period of the registration without concentrating her attention on the process that would be performed. The registration of the heart intervals RR succession, measurement of the length, and development of the RR-intervalogram (FIG. 5A) were accomplished using the apparatus.

The RR-intervalogram for volunteer G and the corresponding spectrum are represented in FIGS. 5A and 5B, respectively. The continual model of the VCR according to the first 128 RR-intervals was built. The integral approximation of VCR with a spectral decomposition of the received succession of RR-intervals, division of the spectrogram into frequency slots of VLF (0.0033-0.04 Hertz), LF (0.04-0.15 Hertz), and HF (0.15-0.4 Hertz) were fulfilled, including measurement of total capacity of the spectrum W_{O} and capacities in the designated frequency intervals W_{VLF}, W_{LF} and W_{HF}.

The results of the indexes' calculation of state of the organism regulation systems of volunteer G according to the declared method and prototype, as well as the average normative values for women of the corresponding age, are given below for comparison:

| | | Average for women of |
|---|---|---|
| Declared method | Prototype | the given age |
| TP = 1468 | 1534 | 2500-4000 |
| LF/HF = 0.18 | 0.2 | 1.0-1.5 |
| VLF/LF = 1.25 | 0.7 | 0.8-1.2 |

The results according to the declared method and prototype do not differ considerably and this is stipulated by quasistationarity of the record when an application of the prototype is allowed to receive correct results.

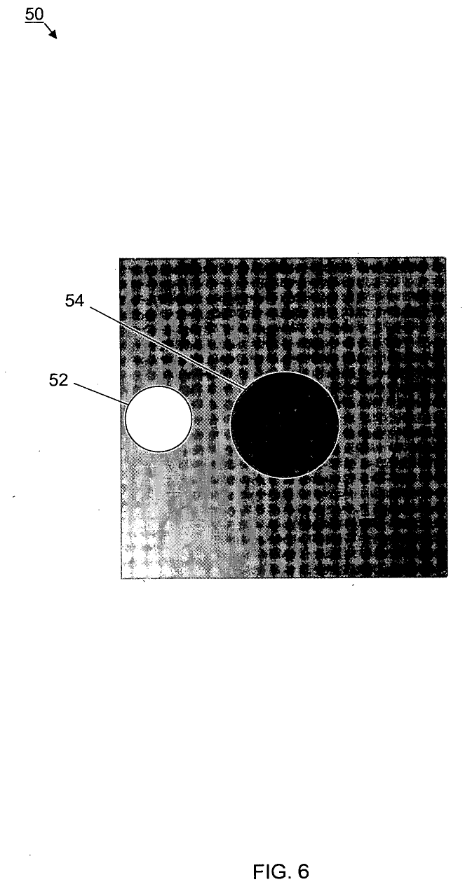

With reference to FIG. 6, the results of the evaluation of volunteer G's regulation state are represented on the phase plane W_{VLF}/W_{LF}−W_{LF}/W_{HF} 50 in the form of a circumference with radius W₀ 52 with moving coordinates of the center i_{X}−ii_{X} and a circumference with radius W_{N} 54 with coordinates of the center i_{N}−ii_{N} which are placed at the computing origin. The circumference with radius W_{N} 54 with coordinates of the center i_{N}−ii_{N} determines the normative position and the regulation level for women of this age. The circumference with radius W₀ 52 with moving coordinates of the center i_{X}−ii_{X} determines the regulation state of volunteer G. The distance between the centers of the circumferences and the correlation of the radiuses of the circumferences show the degree of deviation of the volunteer G's regulation state from the age norms. The results received according to the declared method prove the decrease of regulation capacity with the capacity predominance of W_{VLF} over W_{HF}.

### EXAMPLE 3

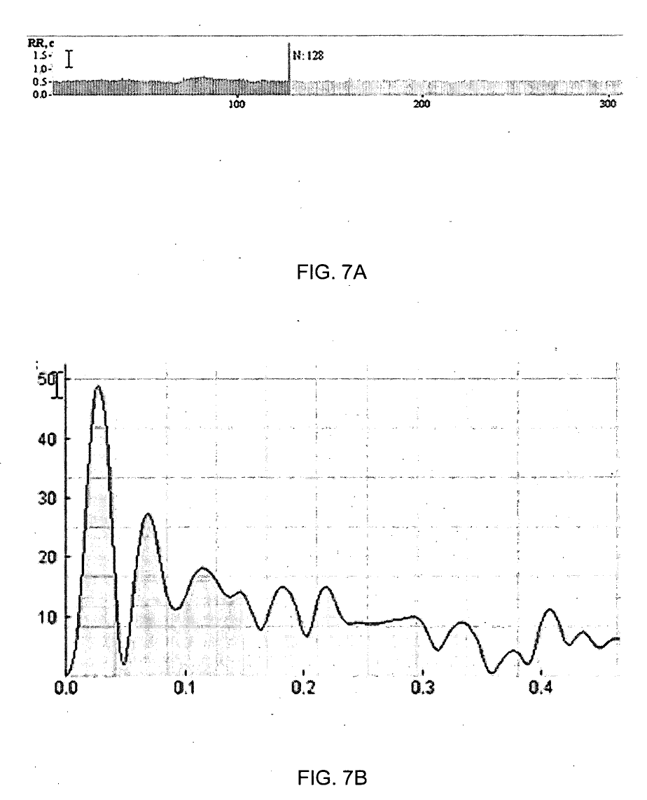

A male that was 24 years old and from Base (folder) Antonan Sasha, registration 11-40, was identified as patient or volunteer A. He was instructed to sit in an armchair in a relaxed position, leaning against the back of the armchair. Two electrodes of the heart intervals RR registration device are placed on his right and left forearms. The “negative” electrode was placed on the left forearm and the “positive” electrode was placed on the right forearm. Next, volunteer A was asked to remain still in this position for the whole period of the registration without concentrating his attention on the process that would be performed. The registration of the heart intervals RR succession, measurement of the length, and development of the RR-intervalogram (FIG. 7A) were accomplished using the apparatus. The RR-intervalogram for volunteer A and the corresponding spectrum are represented in FIGS. 7A and 7B, respectively.

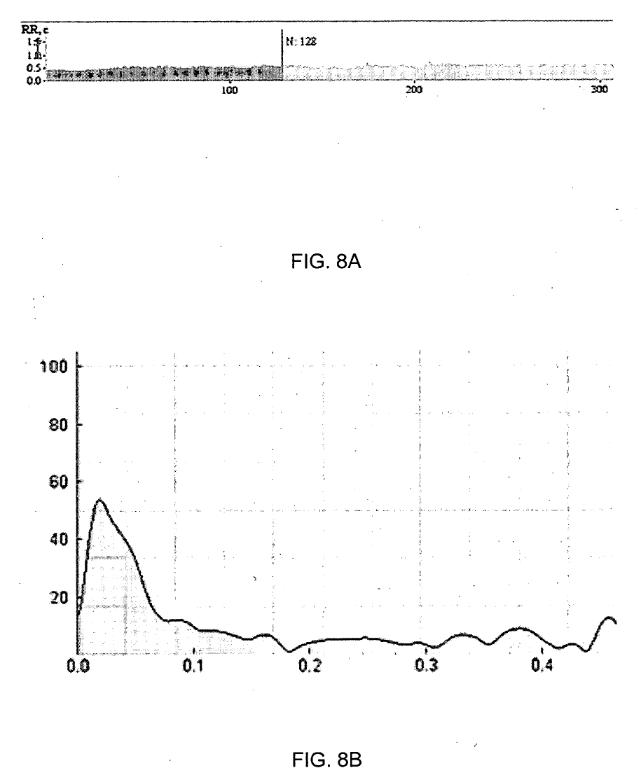

Next, volunteer A was asked to do physical exercises (e.g., squats) in moderate tempo for a period of about three minutes. After the physical exercises were completed, the examination procedure was repeated. The second RR-intervalogram for volunteer A after the physical exercises and the corresponding spectrum are represented in FIGS. 8A and 8B, respectively.

The results of the measurements of the corresponding indices before and after the physical exercises, as well as the average indices for men of the corresponding age, are given below for comparison:

| | | Average for men of |
|---|---|---|
| Before test | After test | the given age |
| TP = 877 | 2839 | 2000-3500 |
| LF/HF = 1.14 | 1.0 | 1.3-1.7 |
| VLF/LF = 15 | 7.3 | 1.0-1.4 |

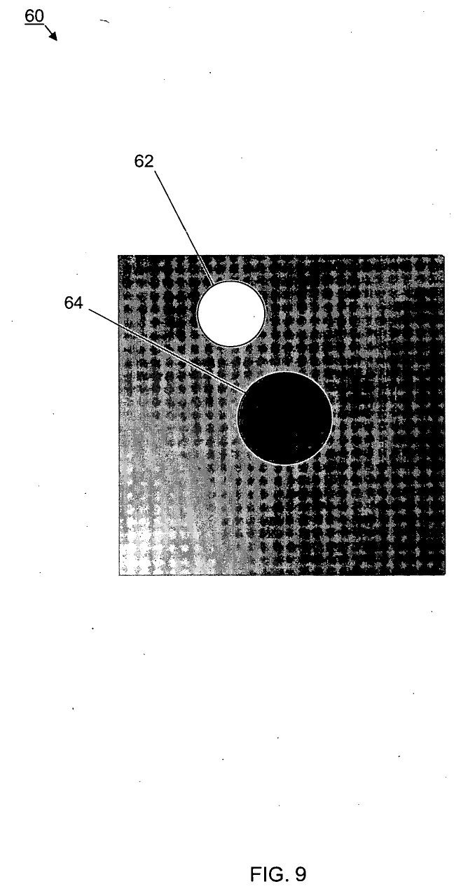

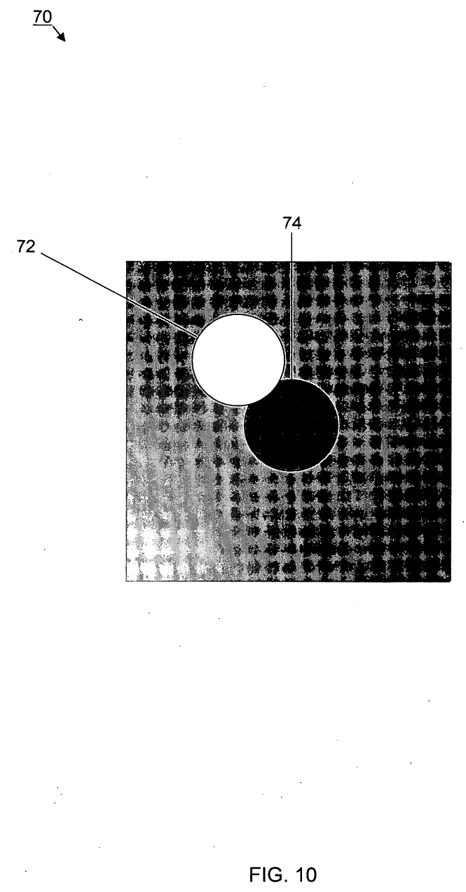

With reference to FIGS. 9 and 10, the evaluation results of the regulation state of volunteer A before and after the physical exercises are represented on the phase plane W_{VLF}/W_{LF}−W_{LF}/W_{HF} 60, 70 in the form of a circumference with radius W₀ 62, 72 with moving coordinates of the center i_{X}−ii_{X} and a circumference with radius W_{N} 64, 74 with the coordinates of the center i_{N}−ii_{N} which are placed at the computing origin. The circumference with radius W_{N} 64, 74 with the coordinates of the center i_{N}−ii_{N} determines the normative position and the level of regulation for men of this age. The circumference with radius W₀ 62, 72 with the moving coordinates of the center i_{X}−ii_{X} determines the state of regulation of volunteer A. FIG. 10 shows the positive reaction of the regulatory systems to the physical exercises. Note that the regulation capacity rose sharply, the ratio of VLF/LF went down, and the ratio of LF/HF stayed without changes.

### EXAMPLE 4

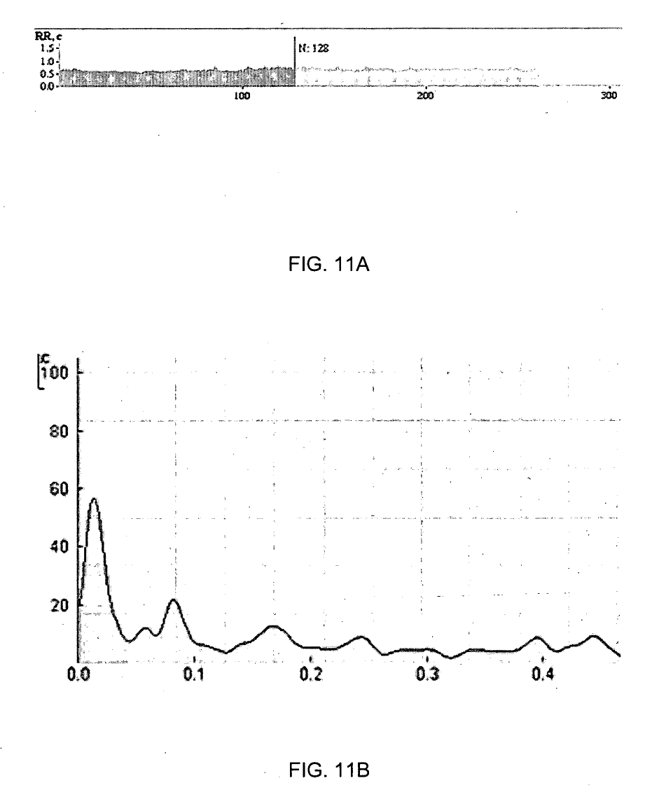

A male that was 20 years old and from Base (folder) Melnik Mitia, registration 12-31, was identified as patient or volunteer M. He was instructed to sit in an armchair in a relaxed position, leaning against the back of the armchair. Two electrodes of the heart intervals RR registration device were placed on his right and left forearms. The “negative” electrode was placed on the left forearm and the “positive” electrode was placed on the right forearm. The patient was asked to remain still in this position for the whole period of the registration without concentrating his attention on the process that would be performed. The registration of the heart intervals RR succession, measurement of the length, and development of the RR-intervalogram (FIG. 11A) were accomplished using the apparatus. The RR-intervalogram for volunteer M and the corresponding spectrum are represented in FIGS. 11A and 11B, respectively.

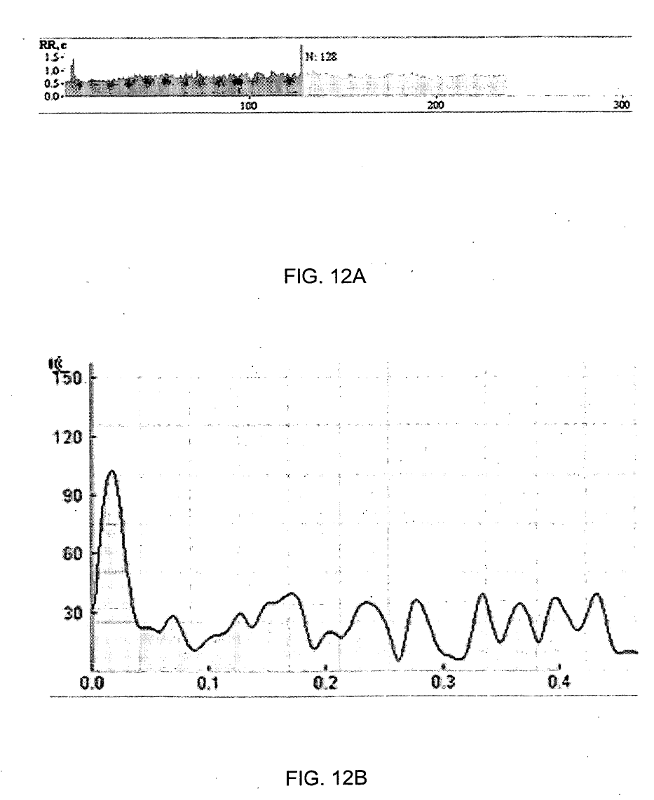

Next, volunteer M was asked to listen to a musical composition (e.g., rock music) for about five minutes. After the listening was completed, the examination procedure was repeated. The second RR-intervalogram for, volunteer M after the listening and the corresponding spectrum are represented in FIGS. 12A and 12B, respectively.

The results of the measurements of the corresponding indices before and after the listening to the musical composition, as well as the average indices for men of the corresponding age, are given below for comparison:

| | | Average for men of |
|---|---|---|
| Before test | After test | the given age |
| TP = 3184 | 7981 | 2000-3500 |
| LF/HF = 1.02 | 0.23 | 1.3-1.7 |
| VLF/LF = 4.7 | 4.3 | 1.0-1.4 |

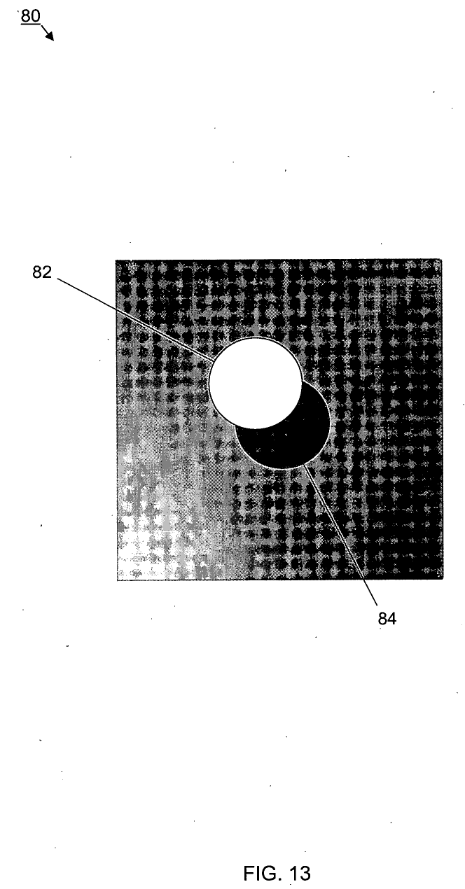

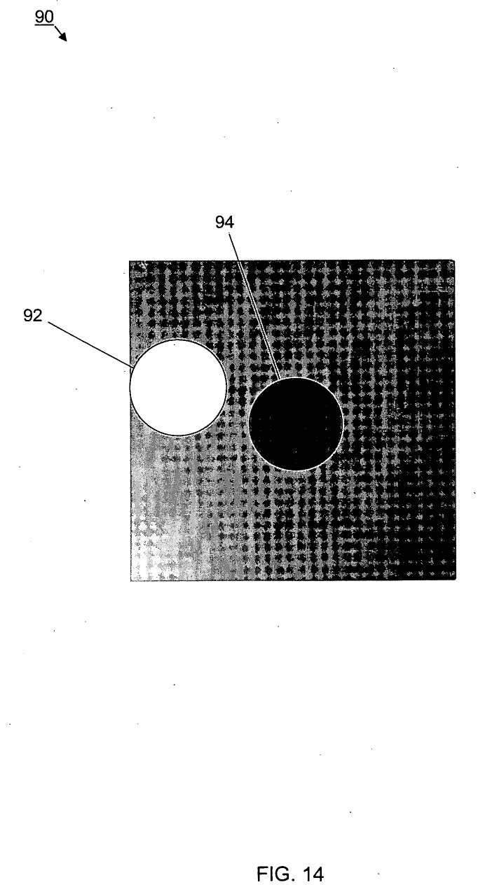

With reference to FIGS. 13 and 14, the results of the regulation state evaluation of the volunteer M before and after the listening to the musical composition are represented on the phase plane W_{VLF}/W_{LF}−W_{LF}/W_{HF} 80, 90 in the form of a circumference with radius W₀ 82, 92 with moving coordinates of the center i_{X}−ii_{X} and a circumference with radius W_{N} 84, 94 with coordinates of the center i_{N}−ii_{N} which are placed at the computing origin. The circumference with radius W_{N} 84, 94 with the coordinates of the center i_{N}−ii_{N} determines the normative position and the regulation level for men of this age. The circumference with radius W₀ 82, 92 with moving coordinates of the center i_{X}−ii_{X} determines the regulation state of the volunteer M.

Comparing the results in FIGS. 13 and 14, it is easy to see that, after the listening to the musical composition, the circumference with radius W₀ 92 exceeds the normative W_{N} 94 substantially and the distance between their coordinates increases, and changes the position of the circumference with radius W₀ 92 on the phase plane. Thus, the listening to the musical composition influences the organism's regulatory systems negatively.

### EXAMPLE 5

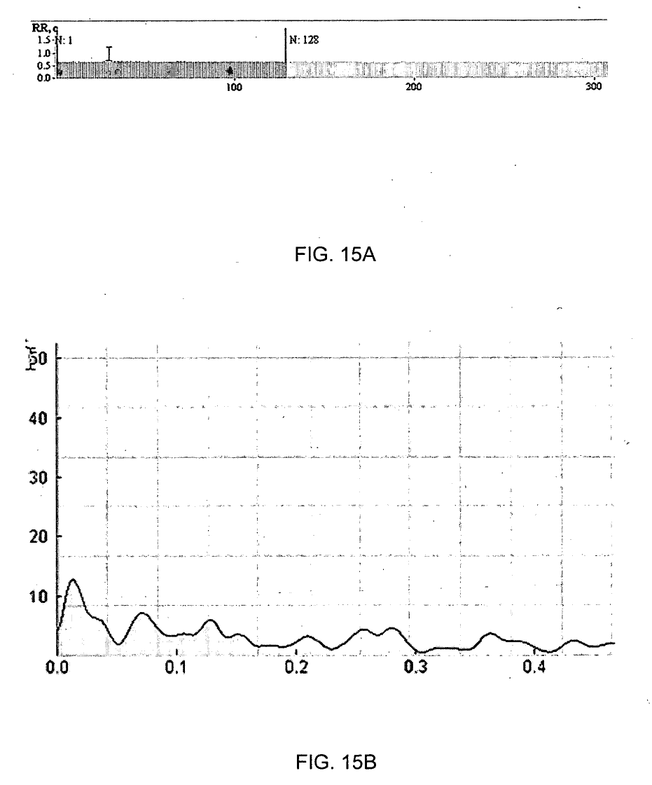

A female that was 64 years old and from Base (1) Kharkov Oblast Hospital—Doroshenko, registration 18-30, was identified as patient or volunteer D. She was instructed to sit in an armchair in a relaxed position, leaning against the back of the armchair. Two electrodes of the heart intervals RR registration device were placed on her right and left forearms. The “negative” electrode was placed on the left forearm and the “positive” electrode was placed on the right forearm. Next, volunteer D was asked to remain still in this position for the whole period of the registration without concentrating her attention on the process that would be performed. The registration of the heart intervals RR succession, measurement of the length, and development of the RR-intervalogram (FIG. 15A) were accomplished using the apparatus. The RR-intervalogram for volunteer D and the corresponding spectrum are represented in FIGS. 15A and 15B, respectively.

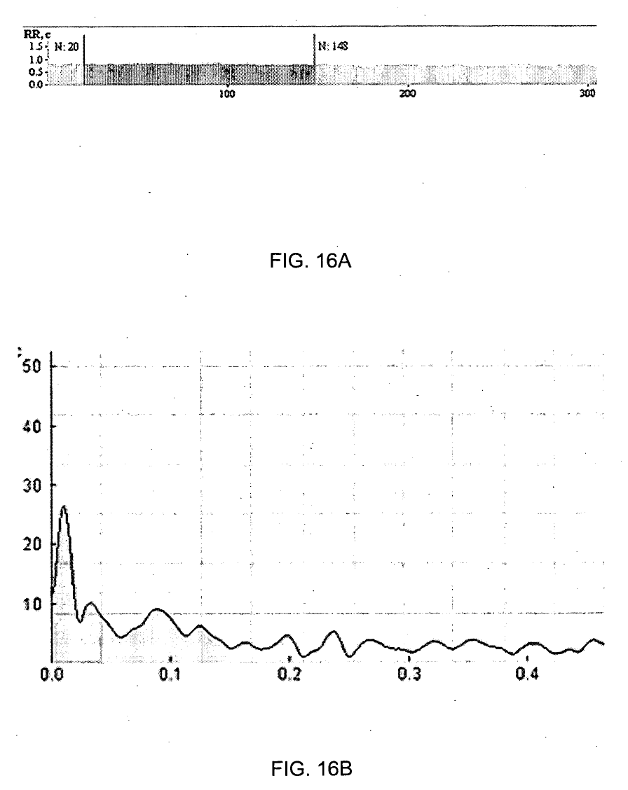

Next, volunteer D was asked to breath with frequency of about twelve cycles per minute without modulation for a period of about five minutes. After the breathing without modulation was completed, the examination procedure was repeated. The second RR-intervalogram for volunteer D after the breathing without modulation and the corresponding spectrum are represented in FIGS. 16A and 16B, respectively.

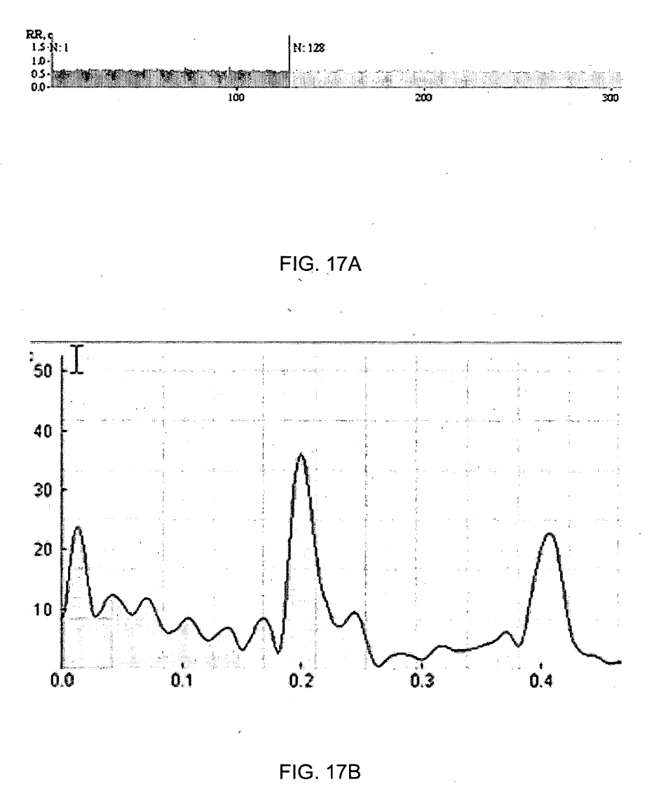

In about five minutes, volunteer D was asked to repeat the breathing again at a frequency of about twelve cycles per minute for a period of about five minutes, but with modulation this time. After the breathing with modulation was completed, the examination procedure was repeated again. The third RR-intervalogram for volunteer D after the breathing with modulation and the corresponding spectrum are represented in FIGS. 17A and 17B, respectively.

The results of the measurements of the corresponding indices before and after the controlled breathing (without and with modulation), as well as the average indices for women of the corresponding age, are given below for comparison:

| | After test | After test | Average for |
|---|---|---|---|
| | without | with | women of |
| Before test | modulation | modulation | the given age |
| TP = 157 | 671 | 890 | 1250-2750 |
| VLF = 67 | 431 | 187 | |
| LF/HF = 1.0 | 1.21 | 0.14 | 1.5-1.9 |
| VLF/LF = 1.5 | 3.3 | 2.2 | 1.2-1.5 |

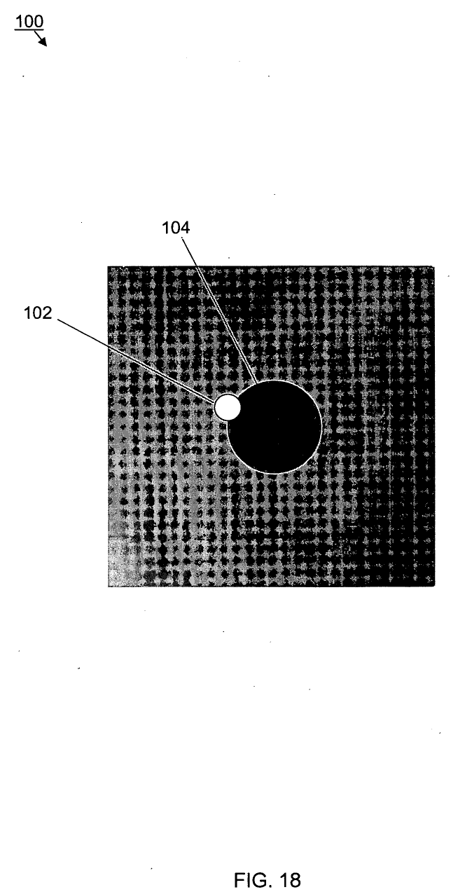

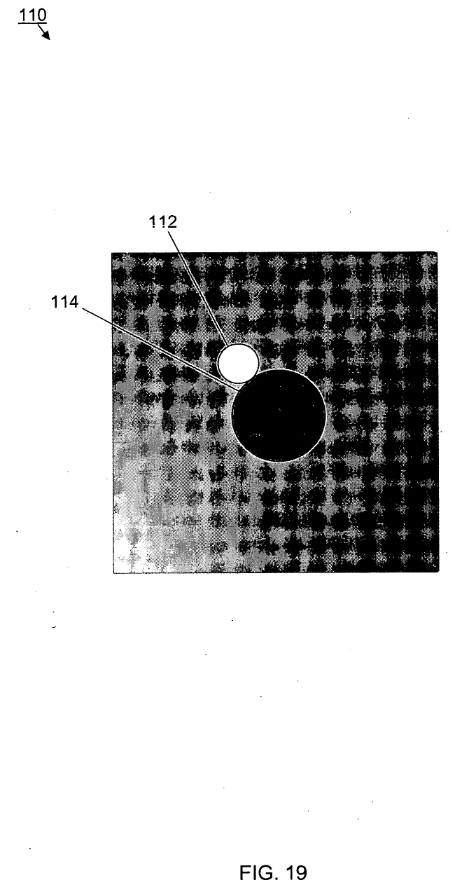

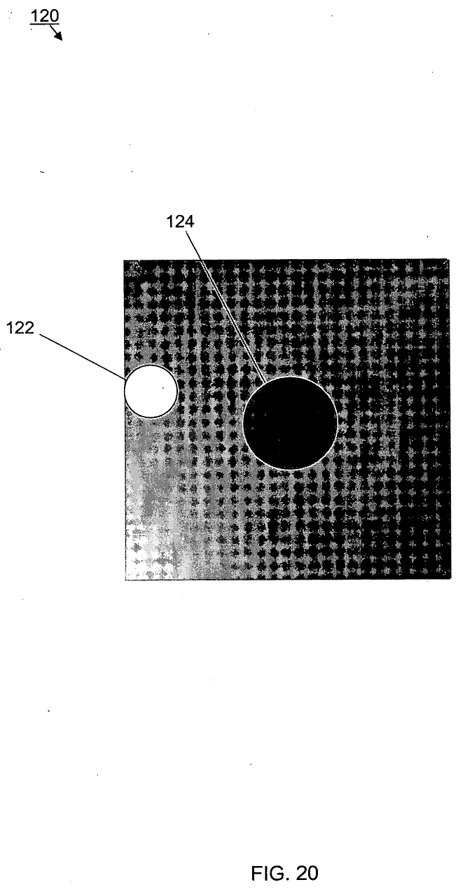

With reference to FIGS. 18-20, the evaluation results of the regulation state of volunteer D before and after the controlled breathing are represented on the phase plane W_{VLF}/W_{LF}−W_{LF}/W_{HF} 100, 110, 120 in the form of a circumference with radius W₀ 102, 112, 122 with the moving coordinates of the center i_{X}−ii_{X} and a circumference with radius W_{N} 104, 114, 124 with the coordinates of the center i_{N}−ii_{N} which are placed at the computing origin. The circumference with radius W_{N} 104, 114, 124 with coordinates of the center i_{N}−ii_{N} determines the normative position and the level of regulation for women of this age. The circumference with radius W₀ 102, 112,122 with moving coordinates of the center i_{X}−ii_{X} determines the state of regulation of volunteer D.

Comparing the results in FIGS. 18-20, it is easy to see that both of the controlled breathing periods made a positive influence on the regulation capacity. However, the controlled breathing with modulation resulted in a larger deviation of the W₀ 122 position with moving coordinates of the center i_{X}−ii_{X} from the W_{N} 124 position with the coordinates of the center i_{N}−ii_{N}. The results with volunteer D show that controlled breathing without modulation has a better positive influence on the regulation than the controlled breathing with modulation.

### EXAMPLE 6

A female that was 34 years old was identified as volunteer S. She was instructed to sit in an armchair in front of the visualization device or monitor in a relaxed position, leaning against the back of the armchair. Two electrodes of the heart intervals RR registration device were placed on her right and left forearms. The “negative” electrode was placed on the left forearm and the “positive” electrode was placed on the right forearm. Next, volunteer S was asked to remain relaxed in this position and to look at the monitor and observe the phase plan. Subsequently, volunteer S will be asked to concentrate on controlling the position of a circumference with radius W₀ with respect to the position of a circumference with radius W_{N} which corresponds to physiological norms and is situated at the center of the phase plane. The registration of the heart intervals RR succession, measurement of the length, and development of the RR-intervalogram were accomplished using the apparatus.

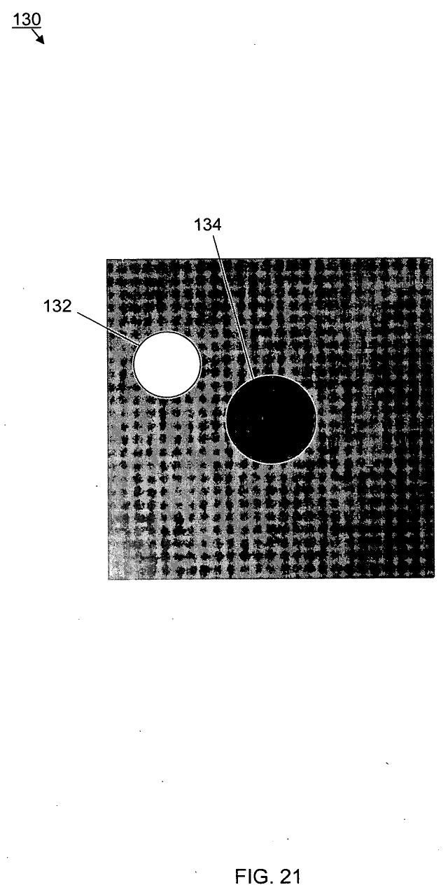

With reference to FIG. 21, the starting position of a circumference with radius W₀ 134 was registered on the monitor with respect to the position of the circumference with radius W_{N} 132 which corresponds to the physiological norms and is situated at the center of the phase plane 130.

Next, volunteer S was asked to put on a headset associated with a CD player, to choose her favorite classical composition from a list of compositions, to turn on the CD player, and, while watching the changes of the radius value and the position of the circumference with radius W₀ 132 with respect to the position of the circumference with radius W_{N} 134 which corresponds to the physiological norms and is situated at the center of the phase plane 130, to change the volume and frequency characteristics of the CD player in such a way that the radius value and the position of the circumference with radius W₀ 132 could maximally approach the radius value and the position of the circumference with radius W_{N} 134 which corresponds to the physiological norms and is situated at the center of the phase plane 130.

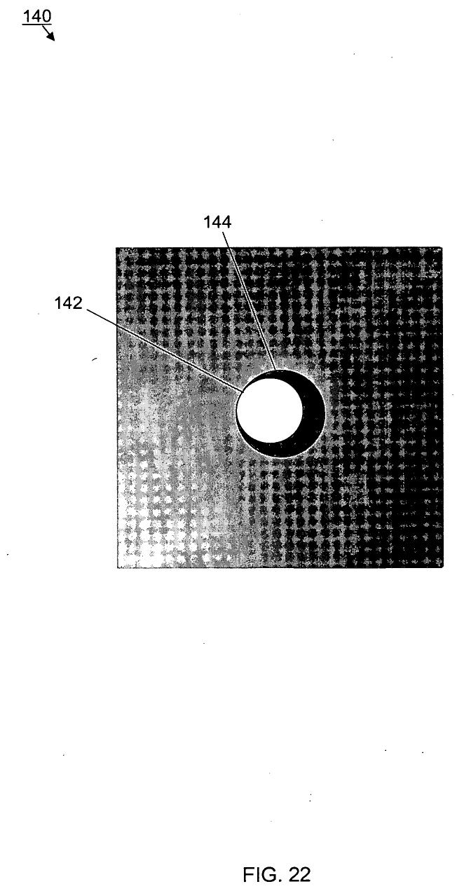

With reference to FIG. 22, the best results of the regulation state of volunteer S was reached on about the fifth minute of the test and remained generally the same during further listening to the musical composition at the same volume and frequency characteristics of the CD player. The diagram shows the phase plane W_{VLF}/W_{LF}−W_{LF}/W_{HF} 140 in the form of a circumference with radius W₀ 142 with the moving coordinates of the center i_{X}−ii_{X} and a circumference with radius W_{N} 144 with the coordinates of the center i_{N}−ii_{N} which are placed at the computing origin.

Next, the volunteer was instructed to listen to some other classical composition from the list and to repeat the test and examination procedure.

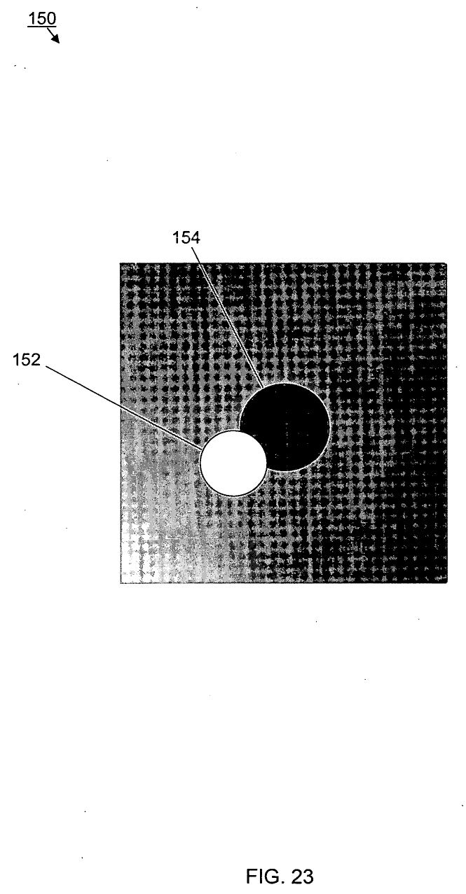

With reference to FIG. 23; the best results of the regulation state of volunteer S in the second procedure shows the phase plane W_{VLF}/W_{LF}−W_{LF}/W_{HF} 150 in the form of a circumference with radius W₀ 152 with the moving coordinates of the center i_{X}−ii_{X} and a circumference with radius W_{N} 154 with the coordinates of the center i_{N}−ii_{N} which are placed at the computing origin.

With reference to FIGS. 21-23, a comparison of the results proves that the better positive result was reached when volunteer S chose her favorite classical composition.

### EXAMPLE 7

A male that was 22 years old was identified as volunteer Z. He was instructed to sit in a chair in a comfortable position, to put his shanks in a bathtub with running water at a temperature of 28 degrees Celsius, and to look at the visualization device or monitor. Two electrodes of the heart intervals RR registration device were placed on his right and left forearms. The “negative” electrode was placed on the left forearm and the “positive” electrode was placed on the right forearm. Next, volunteer Z was asked to watch the position of a circumference with radius W₀ on the monitor with respect to the position of a circumference with radius W_{N} which corresponds to the physiological norms and is situated at the center of the phase plane.

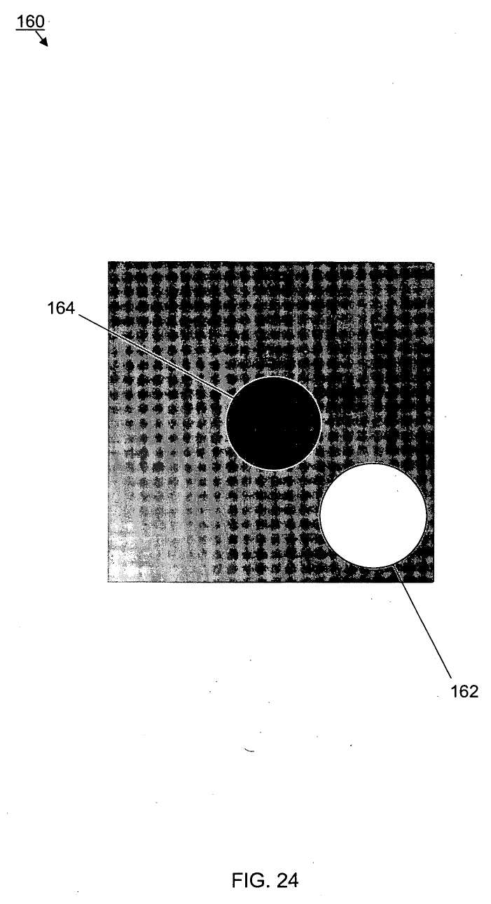

With reference to FIG. 24, the starting position of the circumference with radius W₀ 162 is registered on the monitor with respect to the position of the circumference with radius W_{N} 164 which corresponds to the physiological norms and is situated at the center of the phase plane 160.

Next, volunteer Z was instructed to watch the changes of the radius and of the position of the circumference with radius W₀ 162 during cyclic water temperature changes ranging from 18 to 40 degrees Celsius and to identify such a frequency mode when the radius W₀ 162 maximally approaches the radius W_{N} 164 in value and the circumference W₀ 162 maximally approaches the position of the circumference W_{N} 164.

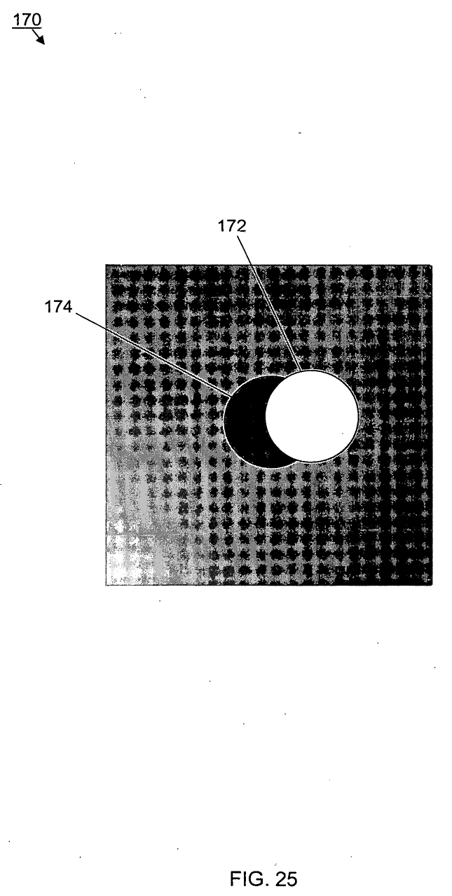

With reference to FIG. 25, the best results of the regulation state of volunteer Z in the second procedure shows the phase plane W_{VLF}/W_{LF}−W_{LF}/W_{HF} 170 in the form of a circumference with radius W₀ 172 with the moving coordinates of the center i_{X}−ii_{X} and a circumference with radius W_{N} 174 with the coordinlates of the center i_{N}−ii_{N} which are placed at the computing origin.

With reference to FIGS. 24 and 25, a comparison of the results proves that a positive result was achieved in the second procedure as volunteer Z performed the given task.

Test 8

A male that was 22 years old was identified as volunteer Zₕ. He was instructed to stand in front of the visualization device or monitor in a comfortable position, facing the monitor. Two electrodes of the heart intervals RR registration device were placed on his right and left forearms. The “negative” electrode was placed on the left forearm and the “positive” electrode was placed on the right forearm. Next, volunteer Zₕ was asked to watch the position of a circumference with radius W₀ on the monitor with respect to the position of a circumference with radius W_{N} which corresponds to the physiological norms and is situated at the center of the phase plane.

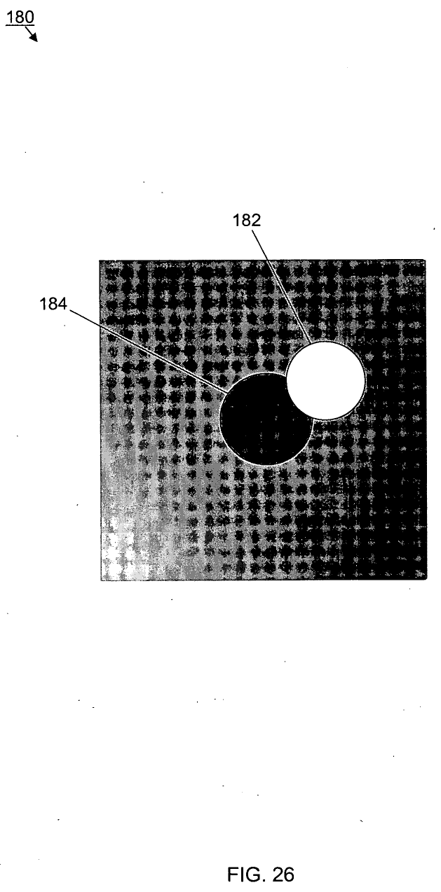

With reference to FIG. 26, the starting position of the circumference with radius W₀ 182 is registered on the monitor with respect to the position of the circumference with radius W_{N} 184 which corresponds to the physiological norms and is situated at the center of the phase plane 180.

Next, volunteer Zₕ was instructed to begin walking in place, to watch the changes of the radius and position of the circumference with radius W₀ 182, and, while changing the pace of the walking, to identify a frequency mode when the radius W₀ 182 maximally approaches the radius W_{N} 184 in value and the circumference W₀ 182 maximally approaches the position of the circumference W_{N} 184.

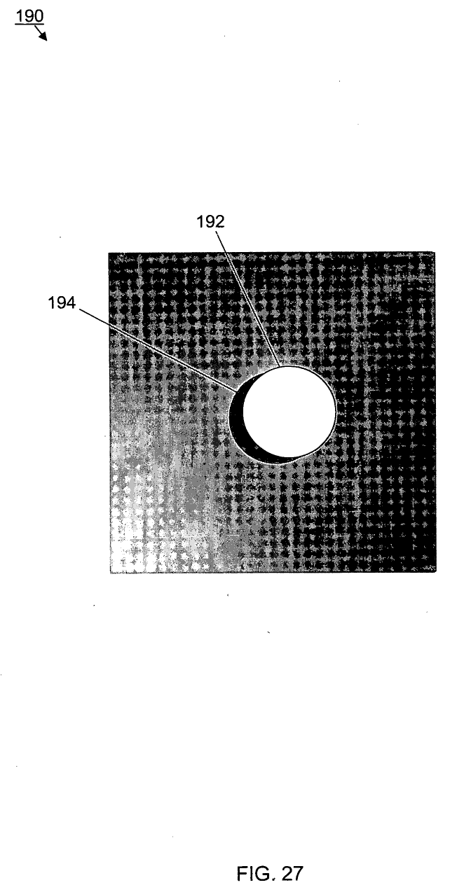

With reference to FIG. 27, the best results of the regulation state of volunteer Zₕ in the procedure with walking in place at different paces shows the phase plane W_{VLF}/W_{LF}−W_{LF}/W_{HF} 190 in the form of a circumference with radius W₀ 192 with the moving coordinates of the center i_{X}−ii_{X} and a circumference with radius W_{N} 194 with the coordinates of the center i_{N}−ii_{N} which are placed at the computing origin.

With reference to FIGS. 26 and 27, a comparison of the results proves that a positive result was achieved in the procedure with walking in place at different paces as volunteer Zₕ performed the given task.

The exemplary embodiments have been described with reference to the preferred embodiments. Obviously, modifications and alterations will occur to others upon reading and understanding the preceding detailed description. It is intended that the exemplary embodiments be construed as including all such modifications and alterations insofar as they come within the scope of the appended claims or the equivalents thereof.

1. A method for integral evaluation of a state of a regulation system of a subject organism, including:
a) registering a heart interval RR for the subject organism;
b) measuring a succession of heart intervals RR associated with the registered heart interval RR to determine an integral approximation of a heart rate variability for the subject organism;
c) performing a spectral decomposition on the succession of heart intervals RR to develop a spectrogram;
d) fragmenting the spectrogram Into low frequency, medium frequency, and high frequency ranges; and
e) determining a first state of the regulation system of the subject organism by determining an index based at least in part on a capacity of the designated frequency ranges for the subject organism and corresponding predetermined average capacities of the designated frequency ranges for a like organism.

2. The method set forth in claim 1 wherein the integral approximation of the heart rate variability is based at least in part on an equation:
$$\overline{R} {\overline{R}}_{j} = {M}_{RR} + A sin ( \frac{2 π}{{M}_{RR}} R {R}_{j} ) ;$$
where \overline{R}\overline{R}ⱼ is a model value of heart interval length, RRⱼ is a measured value of heart interval length, M_{RR} is a medium value of heart interval length, and A is a coefficient that minimizes the deviation of \overline{R}\overline{R}ⱼ from RRⱼ.

3. The method set forth in claim 1 wherein the spectral decomposition includes identification of a Fourier line with a coefficient for k members of the Fourier line and is based at least in part on first equation:
$${a}_{k} = \frac{A {M}_{RR}}{π} {\sum }_{N} ( \frac{cos ( \frac{2 π k}{{T}^{{τ}_{j + 1}}} - \frac{2 π R {R}_{j}}{{M}_{R R}} )}{2 ( k {M}_{R R} - T )} + \\ \frac{cos ( \frac{2 π k}{{T}^{{τ}_{j + 1}}} + \frac{2 π R {R}_{j}}{{M}_{R R}} )}{2 ( k {M}_{R R} + T )} - \frac{T cos ( \frac{2 π k}{{T}^{{τ}_{j}}} )}{( k {M}_{R R} + T ) ( k {M}_{R R} - T )} )$$
and at least in part on a second equation:
$${b}_{k} = \frac{A {M}_{RR}}{π} {\sum }_{N} ( \frac{sin ( \frac{2 π k}{{T}^{{τ}_{j + 1}}} - \frac{2 π {RR}_{j}}{{M}_{RR}} )}{2 ( k {M}_{RR} - T )} + \\ \frac{sin ( \frac{2 π k}{{T}^{{τ}_{j + 1}}} + \frac{2 π {RR}_{j}}{{M}_{RR}} )}{2 ( k {M}_{RR} + T )} - \frac{T sin ( \frac{2 π k}{{T}^{{τ}_{j}}} )}{( k {M}_{RR} + T ) ( k {M}_{RR} - T )} )$$
where RRⱼ is a measured value of heart interval length, M_{RR} is a medium value of heart interval length, T is a temporary interval for which the spectral decomposition is done; N is a quantity of heart intervals in a decomposition section (T=M_{RR} N), τⱼ is a first time and associated with a beginning of interval RRⱼ, and τⱼ₊₁ is a second time and associated an ending of interval RRⱼ.

4. The method set forth in claim 1 wherein the low frequency range is about 0.0033-0.04 Hertz, the medium frequency range is about 0.04-0.15 Hertz, and the high frequency range is about 0.15-0.4 Hertz.

5. The method set forth in claim 1 wherein the capacity of the designated frequency ranges for the subject organism is determined at least in part using a first equation:
W_{O}=W_{VLF}+W_{LF}+W_{HF};
where W_{O} is the total capacity for the subject organism over a frequency spectrum defined by the low, medium, and high frequency ranges, W_{VLF} is the capacity for the subject organism with respect to the low frequency range, W_{LF} is the capacity for the subject organism with respect to the medium frequency range, and W_{HF} is the capacity for the subject organism with respect to the high frequency range; and
wherein the predetermined average capacity of the designated frequency ranges for the like organism is represented at least in part using a second equation:
W_{N}=W_{VLF}+W_{LF}+W_{HF};
where W_{N} is the predetermined average capacity for the like organism over a frequency spectrum defined by the low, medium, and high frequency ranges, W_{VLF} is the predetermined average capacity for the like organism with respect to the low frequency range, W_{LF} is the predetermined average capacity for the like organism with respect to the medium frequency range, and W_{HF} is the predetermined average capacity for the like organism with respect to the high frequency range.

6. The method set forth in claim 5 wherein the index for determining the first state of the regulation system for the subject organism is determined at least in part using an equation:
N=[(i_{X}−i_{N})²+(ii_{X}−ii_{N})²]^{1/2}
where i_{X}=W_{VLF}/(W_{HF}+W_{LF}) for the subject organism, ii_{X}=W_{LF}/W_{HF} for the subject organism, i_{N}=W_{VLF}/(W_{HF}+W_{LF}) for the like organism, and ii_{N}=W_{LF}/W_{HF}—for the like organism.

7. The method set forth in claim 6, further including:
f) displaying the first state of the regulation system of the subject organism on a visualization device in a phase plane represented by W_{VLF}/(W_{HF}+W_{LF})−W_{LF}/W_{HF} and including a first circular form having a radius defined by W₀ with a moving center coordinate defined by i_{X}−ii_{X} and a second circular form having a radius defined by W_{N} with a fixed center coordinate defined by i_{N}−ii_{N}.

8. The method set forth in claim 1, further including:
f) selecting an effect and causing the regulation system of the subject organism to be affected by the effect for a selected time; and
g) repeating a)-e) to determine a second state of the regulation system of the subject organism.

9. The method set forth in claim 8, further including:
h) determining if the effect had a positive affect or a negative affect on the regulation system of the subject organism.

10. The method set forth in claim 8 wherein effect includes at least one of an effect by a pharmaceutical remedy, a thermal effect, an effect by light, an acoustic effect, an effect by food, an effect by drink, an effect by hunger, an effect by thirst, an effect by a gas medium, an effect by a liquid medium, an effect by the subject organism performing a physical exercise, an effect by emotional stress on the subject organism, an effect by the subject organism performing an intellectual exercise.

11. The method set forth in claim 8 wherein g) is performed during at least a portion of f).

12. The method set forth in claim 11 wherein g) and f) are performed until the regulation system of the subject organism is optimally affected in a positive manner by the effect.

13. The method set forth in claim 1, further including:
f) displaying the first state of the regulation system of the subject organism on a visualization device.

14. The method set forth in claim 13, further including:
g) selecting an effect and causing the regulation system of the subject organism to be affected by the effect for a selected time; and
h) repeating a)-f) to determine and display a second state of the regulation system of the subject organism;
wherein h) is performed during at least a portion of g) and the subject organism is instructed to observe the second state of the regulation system displayed on the visualization device and to attempt to optimize the second state of the regulation system.

15. The method set forth in claim 14 wherein the second state of the regulation system is optimized at least in part by the subject organism adjusting one or more parameters associated with the effect during g).

16. An apparatus for integral evaluation of a state of a regulation system of a subject organism, including:
a plurality of sensors adapted to sense heart intervals RR associated with the subject organism; and
a signal processing block in communication with each of the plurality of sensors and adapted to register a heart interval RR for the subject organism, measure a succession of heart intervals RR associated with the registered heart interval RR to determine an integral approximation of a heart rate variability for the subject organism, perform a spectral decomposition on the succession of heart intervals RR to develop a spectrogram, fragment the spectrogram into low frequency, medium frequency, and high frequency ranges, and determine a first state of the regulation system of the subject organism by determining an index based at least in part on a capacity of the designated frequency ranges for the subject organism and corresponding predetermined average capacities of the designated frequency ranges for a like organism.

17. The apparatus set forth in claim 16, further including:
a visualization device in communication with the signal processing block and adapted to display the first state of the regulation system of the subject organism in a display screen.

18. The apparatus set forth in claim 17 wherein the capacity of the designated frequency ranges for the subject organism is determined at least in part using a first equation:
W_{O}=W_{VLF}+W_{LF}+W_{HF};
where W_{O} is the total capacity for the subject organism over a frequency spectrum defined by the low, medium, and high frequency ranges, W_{VLF} is the capacity for the subject organism with respect to the low frequency range, W_{LF} is the capacity for the subject organism with respect to the medium frequency range, and W_{HF} is the capacity for the subject organism with respect to the high frequency range;
wherein the predetermined average capacity of the designated frequency ranges for the like organism is represented at least in part using a second equation:
W_{N}=W_{VLF}+W_{LF}+W_{HF};
where W_{N} is the predetermined average capacity for the like organism over a frequency spectrum defined by the low, medium, and high frequency ranges, W_{VLF} is the predetermined average capacity for the like organism with respect to the low frequency range, W_{LF} is the predetermined average capacity for the like organism with respect to the medium frequency range, and W_{HF} is the predetermined average capacity for the like organism with respect to the high frequency range;
wherein the index for determining the first state of the regulation system for the subject organism is determined at least in part using an equation:
N=[(i_{X}−i_{N})²+(ii_{X}−ii_{N})²]^{1/2}
where i_{X}=W_{VLF}/(W_{HF}+W_{LF}) for the subject organism, ii_{X}=W_{LF}/W_{HF} for the subject organism, i_{N}=W_{VLF}/(W_{HF}+W_{LF}) for the like organism, and ii_{N}=W_{LF}/W_{HF}—for the like organism; and
wherein the first state of the regulation system of the subject organism is represented on the display screen of the visualization device in a phase plane defined by W_{VLF}/(W_{HF}+W_{LF})−W_{LF}/W_{HF} and includes a first circular form having a radius defined by W₀ with a moving center coordinate defined by i_{X}−ii_{X} and a second circular form having a radius defined by W_{N} with a fixed center coordinate defined by i_{N}−ii_{N}.

19. A method for integral evaluation of a state of a regulation system of a subject person, including:
a) registering a heart interval RR for the subject person;
b) measuring a succession of heart intervals RR associated with the registered heart interval RR to determine an integral approximation of a heart rate variability for the subject person;
c) performing a spectral decomposition on the succession of heart intervals RR to develop a spectrogram;
d) fragmenting the spectrogram into low frequency, medium frequency, and high frequency ranges; and
e) determining a first state of the regulation system of the subject person by determining an index based at least in part on a capacity of the designated frequency ranges for the subject person and corresponding predetermined average capacities of the designated frequency ranges for a like person;
wherein the integral approximation of the heart rate variability is based at least in part on an equation:
$$\overline{R} {\overline{R}}_{j} = {M}_{RR} + A sin ( \frac{2 π}{{M}_{RR}} R {R}_{j} ) ;$$
where \overline{R}\overline{R}ⱼ is a model value of heart interval length, RRⱼ is a measured value of heart interval length, M_{RR} is a medium value of heart interval length, and A is a coefficient that minimizes the deviation of \overline{R}\overline{R}ⱼ from RRⱼ;
wherein the spectral decomposition includes identification of a Fourier line with a coefficient for k members of the Fourier line and is based at least in part on first equation:
$${a}_{k} = \frac{A {M}_{RR}}{π} {\sum }_{N} ( \frac{cos ( \frac{2 π k}{{T}^{{τ}_{j + 1}}} - \frac{2 π R {R}_{j}}{{M}_{R R}} )}{2 ( k {M}_{R R} - T )} + \\ \frac{cos ( \frac{2 π k}{{T}^{{τ}_{j + 1}}} + \frac{2 π R {R}_{j}}{{M}_{R R}} )}{2 ( k {M}_{R R} + T )} - \frac{T cos ( \frac{2 π k}{{T}^{{τ}_{j}}} )}{( k {M}_{R R} + T ) ( k {M}_{R R} - T )} )$$
and at least in part on a second equation:
$${b}_{k} = \frac{A {M}_{RR}}{π} {\sum }_{N} ( \frac{sin ( \frac{2 π k}{{T}^{{τ}_{j + 1}}} - \frac{2 π {RR}_{j}}{{M}_{RR}} )}{2 ( k {M}_{RR} - T )} + \\ \frac{sin ( \frac{2 π k}{{T}^{{τ}_{j + 1}}} + \frac{2 π {RR}_{j}}{{M}_{RR}} )}{2 ( k {M}_{RR} + T )} - \frac{T sin ( \frac{2 π k}{{T}^{{τ}_{j}}} )}{( k {M}_{RR} + T ) ( k {M}_{RR} - T )} )$$
where RRⱼ is a measured value of heart interval length, M_{RR} is a medium value of heart interval length, T is a temporary interval for which the spectral decomposition is done; N is a quantity of heart intervals in a decomposition section (T=M_{RR} N), τⱼ is a first time and associated with a beginning of interval RRⱼ, and τⱼ₊₁ is a second time and associated an ending of interval RRⱼ;
wherein the capacity of the designated frequency ranges for the subject person is determined at least in part using a first equation:
W_{O}=W_{VLF}+W_{LF}+W_{HF};
where W_{O} is the total capacity for the subject person over a frequency spectrum defined by the low, medium, and high frequency ranges, W_{VLF} is the capacity for the subject person with respect to the low frequency range, W_{LF} is the capacity for the subject person with respect to the medium frequency range, and W_{HF} is the capacity for the subject person with respect to the high frequency range;
wherein the predetermined average capacity of the designated frequency ranges for the like person is represented at least in part using a second equation:
W_{N}=W_{VLF}+W_{LF}+W_{HF};
where W_{N} is the predetermined average capacity for the like person over a frequency spectrum defined by the low, medium, and high frequency ranges, W_{VLF} is the predetermined average capacity for the like person with respect to the low frequency range, W_{LF} is the predetermined average capacity for the like person with respect to the medium frequency range, and W_{HF} is the predetermined average capacity for the like person with respect to the high frequency range; and
wherein the index for determining the first state of the regulation system for the subject person is determined at least in part using an equation:
N=[(i_{X}−i_{N})²+(ii_{N}−ii_{N})²]^{1/2}
where i_{X}=W_{VLF}/(W_{HF}+W_{LF}) for the subject person, ii_{X}=W_{LF}/W_{HF} for the subject person, i_{N}=W_{VLF}/(W_{HF}+W_{LF}) for the like person, and ii_{N}=W_{LF}/W_{HF}—for the like person.

20. The method set forth in claim 19, further including:
f) displaying the first state of the regulation system of the subject person on a visualization device in a phase plane represented by W_{VLF}/(W_{HF}+W_{LF})−W_{LF}/W_{HF} and including a first circular form having a radius defined by W₀ with a moving center coordinate defined by i_{X}−ii_{X} and a second circular form having a radius defined by W_{N} with a fixed center coordinate defined by i_{N}−ii_{N}.
